(19) United States
(12) Patent Application Publication
(10) Pub. No.: US 2012/0177721 A1
(43) Pub. Date: Jul. 12, 2012

(54) FORMULATION

(76) Inventors: S. Munir Alam, Durham, NC (US); Barton F. Haynes, Durham, NC (US); Stephen Harrison, Boston, MA (US); Bing Chen, Chestnut Hill, MA (US)

(21) Appl. No.: 13/262,661

(22) PCT Filed: Apr. 5, 2010
(86) PCT No.: PCT/US2010/001018

§ 371 Date: Jan. 23, 2012

Related U.S. Application Data
(60) Provisional application No. 61/166,648, filed on Apr. 3, 2009.

(51) Int. Cl. A61K 39/385; A61K 38/21; A61P 31/18; A61K 9/127

(52) U.S. Cl. 424/450; 424/196.11; 424/85.7

(57) ABSTRACT

The present invention relates, in general, to a formulation suitable for use in inducing anti-HIV-1 antibodies and, in particular, to a formulation comprising a prehairpin intermediate form of HIV-1 envelope gp41 linked to a liposome. The invention also relates to methods of inducing broadly neutralizing anti-HIV-1 antibodies using such a formulation.

This application claims priority from U.S. Provisional Application No. 61/166,648, filed Apr. 3, 2009, the entire content of which is incorporated herein by reference.

This invention was made with government support under Grant No. AI 067854 awarded by the National Institutes of Health. The government has certain rights in the invention.

### TECHNICAL FIELD

The present invention relates, in general, to a formulation suitable for use in inducing anti-HIV-1 antibodies and, in particular, to a formulation comprising a prehairpin intermediate form of HIV-1 envelope gp41 linked to a liposome. The invention also relates to methods of inducing broadly neutralizing anti-HIV-1 antibodies using such a formulation.

### BACKGROUND

HIV-1 infection generally induces a strong antibody response to the envelope glycoprotein [trimeric (gp160)₃, cleaved to (gp120/gp41)₃], the sole antigen on the virion surface. Most induced antibodies are ineffective in preventing infection, however, because they are either nonneutralizing or narrowly isolate-specific, and the virus replicates so rapidly that ongoing selection of neutralization-resistant mutants allows viral evolution to “keep ahead” of high-affinity antibody production (Wei et al., Nature 422:307-312 (2003)). Moreover, much of the antibody response may be to rearranged or dissociated forms of gp120 and gp41, on which the dominant epitopes may be either in hypervariable loops or in positions occluded on virion-borne envelope trimer. Rare, “broadly neutralizing” antibodies have been detected that recognize one of three relatively conserved regions on the envelope protein: the CD4-binding site (mAb b12) (Burton et al, Science 266:1024-1027 (1994)); carbohydrates on the outer gp120 surface (mAb 2G12) (Trkola et al, J Virol. 70:1100-1108 (1996)); and a segment of the gp41 ectodomain adjacent to the viral membrane (mAbs 2F5 and 4E10) (Cardoso et al, Immunity 22:163-173 (2005); Ofek et al, J Virol. 78:10724-10737 (2004)), often called the “membrane-proximal external region” (MPER).

Fusion of viral and target-cell membranes initiates HIV-1 infection. Conformational changes in gp120 that accompany its binding to receptor (CD4) and coreceptor (e.g., CCR5 or CXCR4) lead to dissociation of gp120 from gp41 and a cascade of refolding' events in the latter (Harrison, Adv Virus Res. 64:231-259 (2005)). In the course of these rearrangements, the N-terminal fusion peptide of gp41 translocates and inserts into the target-cell membrane. A proposed extended conformation of the gp41 ectodomain, with its fusion peptide thus inserted and the transmembrane anchor still in the viral membrane, has been called the “prehairpin intermediate” (Chan et al, Cell 93.681-684 (1998)). It is the target of various fusion inhibitors, including T-20/enfuvirtide, the first approved fusion-inhibiting antiviral drug (Kilby et al, N Eng J Med. 348:2228-2238 (2003)), and the characteristics of the intermediate have been deduced from the properties of these inhibitors or mimicries by short gp41 fragments (Eckert et al, Cell 99:103-115 (1999)). Subsequent rearrangements from the intermediate to the postfusion state of gp41 involve folding back of each of the three chains into a hairpin-like conformation, with two antiparallel a-helices connected by a disulfide-containing loop. This process brings the fusion peptide and transmembrane anchor, and hence the two membranes, close together at the same end of the refolded protein.

Questions presented include where in this sequence of events do neutralizing antibodies intervene, and can any such antibodies neutralize more than a narrow range of isolates. The first step toward answering these questions is the preparation of biochemically homogeneous forms of the HIV envelope glycoprotein with defined and uniform antigenic properties, which include each of the principal states of the gp41 ectodomain: the prefusion, the prehairpin intermediate, and the postfusion conformations. Dislcosed herein are stable, homogeneous preparations of trimeric HIV-1 envelope protein in relevant states. The present invention results, at least in part, from studies demonstrating that the epitopes for the MPER antibodies, 2F5 and 4E10, are exposed only on the form of the envelope protein designed to mimic the prehairpin intermediate. These results assist in explaining the rarity of 2F5- and 4E10-like antibody responses and provide insight into design of an immunogen that can be used to elicit such responses.

### SUMMARY OF THE INVENTION

In general, the present invention relates to a formulation suitable for use in inducing anti-HIV-1 antibodies. More specifically, the invention relates to a formulation comprising a prehairpin intermediate form of HIV-1 envelope gp41 linked to a liposome. The invention also relates to methods of inducing broadly reactive neutralizing anti-HIV-1 antibodies using such a formulation.

Objects and advantages of the present invention will be clear from the description that follows.

### BRIEF DESCRIPTION OF THE DRAWINGS

FIG. 1. The prehairpin intermediate constructs of HIV-1 gp41 (gp41-inter, Frey et al, Proc. Natl. Acad. Sci. 105:3739-3744 (2008)). Segments of HIV-1 Env protein are designated as follows: HR1—heptad repeat 1, HR2—heptad repeat 2, C-C loop—immunodominant loop with disulfide bond, MPER—membrane proximal external region, His6—6 histidine tag, fd—foldon trimerization tag, GCN4—leucine zipper trimerization domain.

FIG. 2. Structures of TLR agonists formulated with liposomes. A schematic picture of the immunogen designs shows the peptide-liposomes containing TLR agonists as adjuvants; TLR4 (Lipid A); TLR9 (oCpG) and TLR7 (R848).

FIG. 3. Conjugation of gp41-inter protein to synthetic liposomes with and without adjuvants. HIV-1 gp41-inter with a short sequence of histidine residues (His6) at the c-terminus end was immobilized on synthetic liposomes containing a nickel-chelating group (N″, N″-bis[carboxymethyl]-L-lysine; nitriloacetic acid, NTA) covalently attached to the lipid molecules (DOGS, 1,2 dioleoyl-sn- glycerol-3-succinyl-NTA-Ni). The bottom figure is an example of the design of gp41-inter liposomes with two different TLR ligands.

FIGS. 4A-4C. Interaction of 2F5 mAb with MPER peptide-liposomes conjugated to TLR adjuvants. FIG. 4A shows strong binding of 2F5 mab to gp41 MPER liposome constructs with Lipid A (200 ug dose equivalent). FIG. 4B shows binding of 2F5 mAb to oCpG (50ug dose equivalent) conjugated gp41 MPER liposomes. FIG. 4C shows binding of 2F5 mAb to R848-conjugated gp41 MPER containing liposomes. In comparison to control liposomes with only TLR adjuvants, strong binding of 2F5 mAb was observed to each of the gp41 MPER-adjuvant liposomal contructs. MPER bi-epitope (MPER656-NEQELLELDKWASLWNWFNITNWLWYIK) construct include binding epitopes for both 2F5 and 4E1 mAbs).

FIG. 5. Crystal structures of 2F5 (Ofek et al, J. Virol., 78:10724 (2004)) and 4E10 (Cardoso et al, Immunity 22:163-173 (2005)) and design of mutations in the CDR H3 loop to eliminate binding to lipids and HIV-1 viral membrane.

FIGS. 6A and 6B. Substitution of hydrophobic residues of 4E10 (FIG. 6A) and 2F5 (FIG. 6B) CDR H3 loop disrupt lipid binding and abrogate ability of both mAbs to neutralize HIV-1.

FIG. 7. Design of MPER gp41 prehairpin intermediate—liposomes with multiple TLR ligands. Two combinations of TLR ligands are shown, one construct with TLR4+TLR9 and a second one with TLR9+TLR7/8. These constructs have the potential to provide synergy in B cell responses via dual TLR triggering.

FIG. 8. Encapsulation of Interferon alpha (IFNα) into liposomes with gp41-inter and TLR ligands. Any of the combination of TLR ligands shown in FIG. 5 can be used to construct liposomes with encapsulated soluble IFNα.

FIG. 9. Design of CD40 ligand (CD40L) conjugated gp41-inter liposomes. Either soluble CD40 ligand encapsulated into liposomes (Top panel) or a membrane bound version of CD40L can be incorporated into synthetic liposomes.

FIG. 10. Capture of His tagged gp41-inter on immobilized Ni-NTA liposomes.

FIGS. 11A and 11B. Stable binding of MPER neutralizing mAb 2F5 and 4E10 to gp41-inter anchored to liposomes.

FIG. 12. Status of the hypothesis of regulation of broad neutralizing antibodies by tolerance mechanisms.

### DETAILED DESCRIPTION OF THE INVENTION

The present invention relates to a liposome-based adjuvant conjugate that presents a prehairpin intermediate form of HIV-1 envelope gp41, and to a method of inducing neutralizing anti-HIV-1 antibodies in a subject (e.g., a human subject) using same. Suitable neutralizing antigens include gp41 MPER epitope peptides in the form of a gp41 hairpin intermediate construct (or variants thereof (e.g., a

L669S variant of gp41 hairpin intermediate—see U.S. Provisional Appln. No. 61/166,625)). (Shen et al, J. Virology 83: 3617-25 (2009).)

Liposomes suitable for use in the invention include, but are not limited to, those comprising POPC, POPE, DMPA (or sphingomyelin (SM)), lysophosphorylcholine, phosphatidylserine, and cholesterol (Ch). While optimum ratios can be determined by one skilled in the art, examples include POPC:POPE (or POPS):SM:Ch or POPC:POPE (or POPS):DMPA:Ch at ratios of 45:25:20:10. Alternative formulations of liposomes that can be used include DMPC (1,2-dimyristoyl-sn-glycero-3-phosphocholine) (or lysophosphorylcholine), cholesterol (Ch) and DMPG (1,2-dimyristoyl-sn-glycero-3-phoshpho-rac-(1-glycerol) formulated at a molar ratio of 9:7.5:1 (Wassef et al, ImmunoMethods 4:217-222 (1994); Alving et al, G. Gregoriadis (ed.), Liposome technology 2^{nd} ed., vol. III CRC Press, Inc., Boca Raton, Fla. (1993); Richards et al, Infect. Immun. 66(6):285902865 (1998)). The above-described lipid compositions can be complexed with lipid A and used as an immunogen to induce antibody responses against phospholipids (Schuster et al, J. Immunol. 122:900-905 (1979)). A preferred formulation comprises POPC:POPS:Ch at ratios of 60:30:10 complexed with lipid A according to Schuster et al, J. Immunol. 122:900-905 (1979).

In accordance with the invention, immune response enhancing TLR ligands, for example, monophosphorylipid A (MPL-A, TLR4 ligand), oligo CpG (TLR 9 ligand) and R-848 (TLR 7/8 ligand), can be formulated either individually or in combination into the above-described liposomes conjugates. A preferred combination of TLR agonists comprises oCpG (TLR9) (Hemni et al, Nature 408:740-745 (2004)) and R848 (TLR7/8) (Hemni et al, Nat. Immunol. 3:196-200 (2002)).

Additional designs of constructs of the invention include MPER prehairpin intermediate-liposome encapsulated with the cytokine interferon (IFN)-α and either encapsulated or membrane bound CD40 ligand. Two broadly neutralizing gp41 MPER antibodies (2F5, 4E10) bind with high affinity to the gp41 prehairpin intermediate construct (Frey et al, Proc. Natl. Acad. Sci. 105:3739-3744 (2008)). These constructs can be used to modulate B cell tolerance, direct liposomes to certain B cell populations capable of making broadly reactive neutralizing antibodies, and in enhance antibody responses against poorly immunogenic HIV-1 gp41 MPER epitopes.

Autoreactive B cells can be activated by TLR ligands through a mechanism dependent on dual engagement of the B cell receptor (BCR) and TLR (Leadbetter et al, Nature 416:603 (2002); Marshak-Rothstein et al, Annu. Rev. Immunol. 25: 419-41 (2007), Herlands et al, Immunity 29:249-260 (2008), Schlomchik, Immunity 28:18-28 (2008)). In a preferred immunogen design of the instant invention, soluble IFN-α is encapsulated into the MPER prehairpin intermediate-liposome conjugates. IFN-α has been reported to modulate and relax the selectivity for autoreactive B cells by lowering the BCR activation threshold (Uccellini et al, J. Immunol. 181:5875-5884 (2008)). The design of the immunogens results from the observation that lipid reactivity of gp41 MPER antibodies is required for both binding to membrane bound MPER epitopes and in the neutralization of HIV-1.

The B cell subsets that the liposomes can target include any B cell subset capable of making polyreactive antibodies that react with both lipids and the MPER prehairpin intermediates. These B cell subsets include, but are not limited to, the marginal zone IgM+CD27+B cell subset (Weill et al, Annu. Rev. Immunol. 27:267-85 (2009), Li et al, J. Exp. Med 195: 181-188 (2002)), the transitional populations of human B cells (Sims et al, Blood 105:4390-4398 (2005)), and the human equivalent of the B cells that express the human equivalent of the mouse Immunoglobulin (Ig) light chain lambda X (Li et al, Proc. Natl. Acad. Sci. 103:11264-11269 (2006), Witsch et al, J. Exp. Med. 203:1761-1772 (2006)). All of these B cell subsets have the capacity to make multireactive antibodies and, therefore, to make antibodies that have the characteristic of reacting with both lipids and HIV-1 gp41 prehairpin intermediates. That the liposomes have the characteristic of having both lipids and prehairpin intermediate forms of gp41 in them, should result in the selective targeting of these immunogens to the B cells of interest. Because these liposomes can be used to transiently break tolerance of B cells or to target rare B cell subsets, it can be seen that other HIV-1 envelope immunogens, such as deglycosylated envelope preparations, such as described below, can be formulated in the liposomes containing TLR 4 agonists, TLR 7/8 agonists and IFN α.

The deglycosylated JRFL gp140 Env protein and the CD4- binding site mutant gp140 (JRFL APA) have been described in a previous application (see, for example, WO 2008/033500). Deglycosylated env and Env mutated to not bind CD4 so as not to be immunosuppressive can be anchored in the liposomes by incorporating a transmembrane domain and, after solubilizing in detergent, can be reconstituted into synthetic lipsomes. Alternatively, His-tagged (c-terminus end) versions of the Env gp140 can be anchored into liposomes as described for an intermediate form of HIV-1 gp41 (gp41-inter)

Given that many B cell subsets capable of making polyreactive antibodies also bind mammalian DNA, addition of DNA to liposomes can be used to target the immunogens to the responsive B cells.

The liposome-containing formulations of the invention can be administered, for example, by intramuscular, intravenous, intraperitoneal or subcutaneous injection. Additionally, the formulations can be administered via the intranasal route, or intrarectally or vaginally as a suppository-like vehicle. Generally, the liposomes are suspended in an aqueous liquid such as normal saline or phosphate buffered saline pH 7.0. Optimum dosing regimens can be readily determined by one skilled in the art.

Certain aspects of the invention can be described in greater detail in the non-limiting Examples that follows. See also Published PCT Application Nos. WO 2006/110831 and WO 2008/127651, U.S. Published Application Nos. 2008/0031890 and 2008/0057075, U.S. Provisional Application No. 60/960,413 and U.S. application Ser. No. 11/918,219. (See also U.S. Provisional Appln. No. 61/166,625 and U.S. Provisional Application entitled “Mouse Model”, filed Apr. 3, 2009 (Atty Dkt. 01579-1431)).

### EXAMPLE 1

### Description of gp41 MPER Peptide-gp41 Prehairpin Intermediate Conjugates

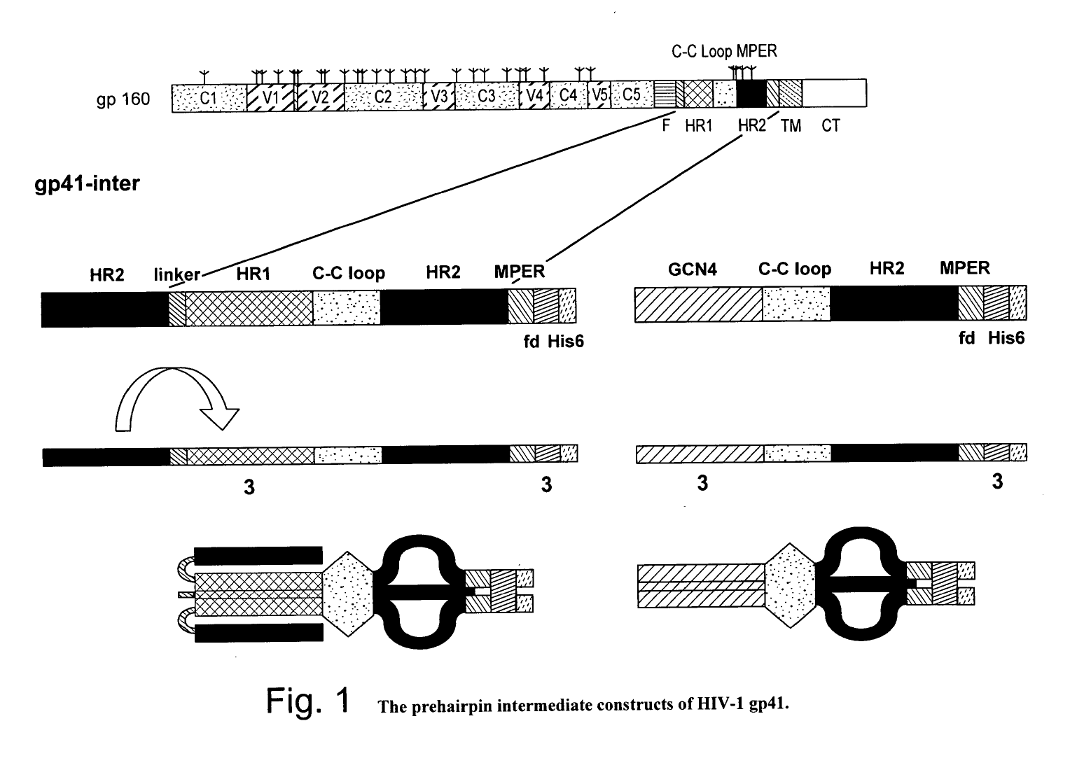

FIG. 1 shows the prehairpin intermediate forms of the HIV-1 gp41 MPER that can be conjugated to synthetic liposomes (Frey et al, Proc. Natl. Acad. Sci. 105:3739-3744 (2008)). To produce biochemically homogeneous forms of additional conformations, two constructs were made that were designed to capture gp41 in the extended, prehairpin intermediate conformation. As shown in FIG. 1, gp41-inter has the following sequence: (HR2)-linker-[HR1-CC loop-HR2-MPER]-(trimerization tag), where HR1 and HR2 are the first and second “heptad repeat” in gp41 (the segments that form helices in the postfusion trimer of hairpins) and the sequence in brackets is essentially the complete gp41 ectodomain, except for the fusion peptide. The “linker” is a short, flexible connector of serines and glycines. When gp41-inter chains trimerize, the N-terminal HR2 segments to form a six-helix bundle with the HR1 segments; the C-terminal HR2 segments, constrained by the trimerization tag, are be unable to do so. The conformation of this construct can be pictured as the prehairpin intermediate captured by an HR2 peptide, such as T-20. gp41-inter was expressed by using sequences from two isolates: 92UG037.8 and HXB2, with foldon and trimeric GCN4, respectively. In both cases, the protein could be expressed in Escherichia coli and refolded in vitro. Controls showed that the N-terminal HR2 segment is required for refolding of bacterially expressed protein and for obtaining soluble, secreted protein from insect cells (data not shown). A similar construct with the gp41 sequence of SIVmac32H and the catalytic subunit of E.

coli aspartate transcarbamoylase as trimer tag (Frey et al, Proc. Natl. Acad. Sci. 105:3739-3744 (2008)) could also be obtained as secreted protein from insect cells (data not shown), indicating that the overall design is robust and independent of the choice of a C-terminal trimerizing element (Frey et al, Proc. Natl. Acad. Sci. 105:3739-3744 (2008), U.S. Provisional Appln. No. 61/032,732).

Purified 92UG-gp41-inter is a monodisperse trimer, stable after multiple rounds of gel-filtration chromatography. Its CD spectrum suggests a mixture of secondary structures. Negative-stain electron microscopy shows rod-like particles, 150 Angstroms in length and ≈45 Angstroms wide. The expected lengths for the N-terminal six-helix bundle and the C-terminal foldon are 75 and 28 Anstroms, respectively. The intervening segment of ≈100 residues (C—C loop, HR2, and MPER) must have a relatively compact fold, to span just 45-50 Angstroms of axial distance (Frey et al, Proc. Natl. Acad. Sci. 105:3739-3744 (2008)).

### Description of gp41 MPER Prehairpin Intermediate-Adjuvant Conjugates

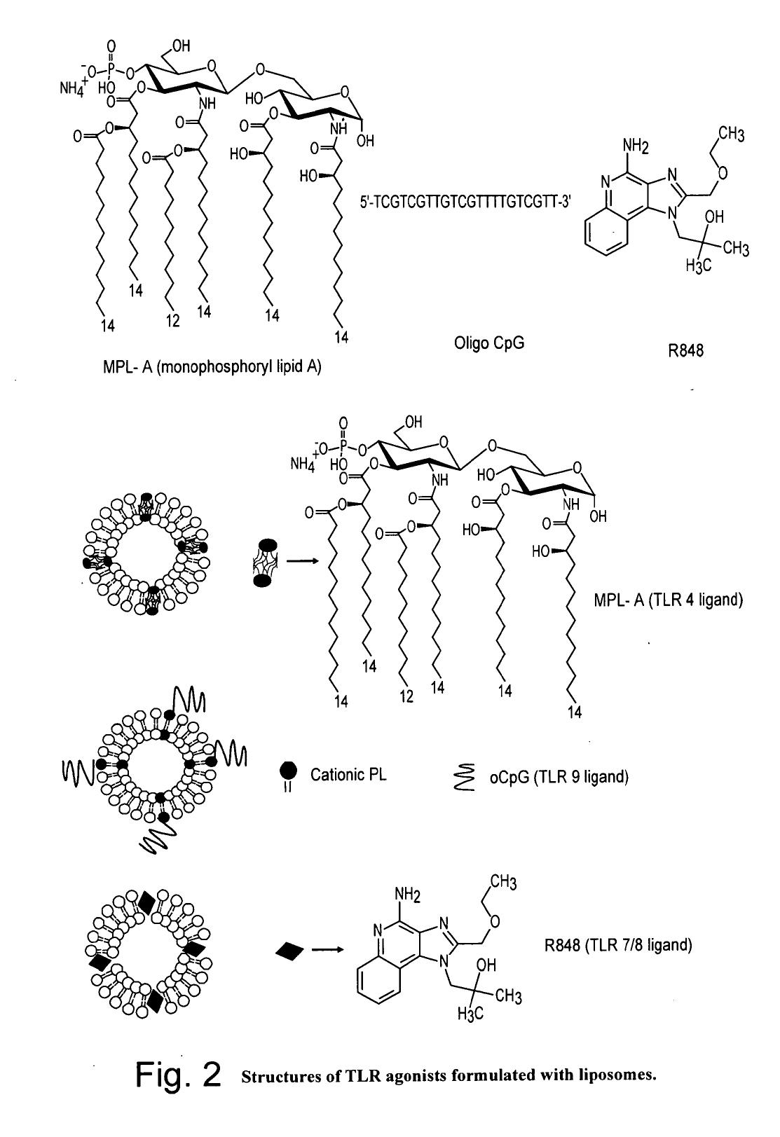

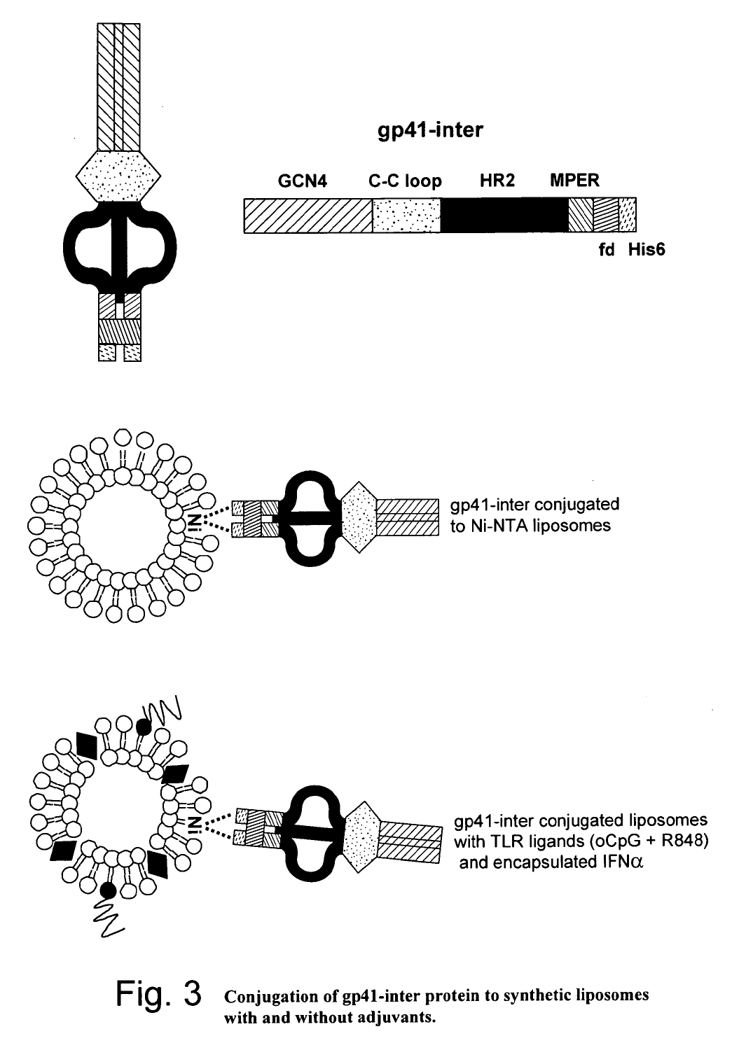

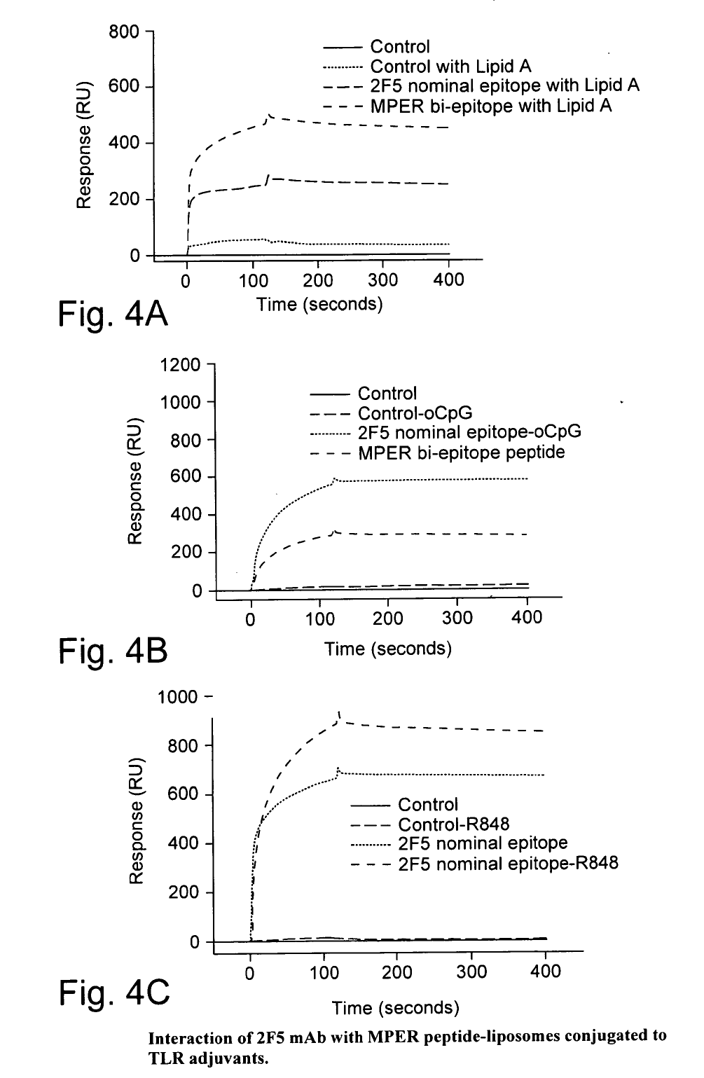

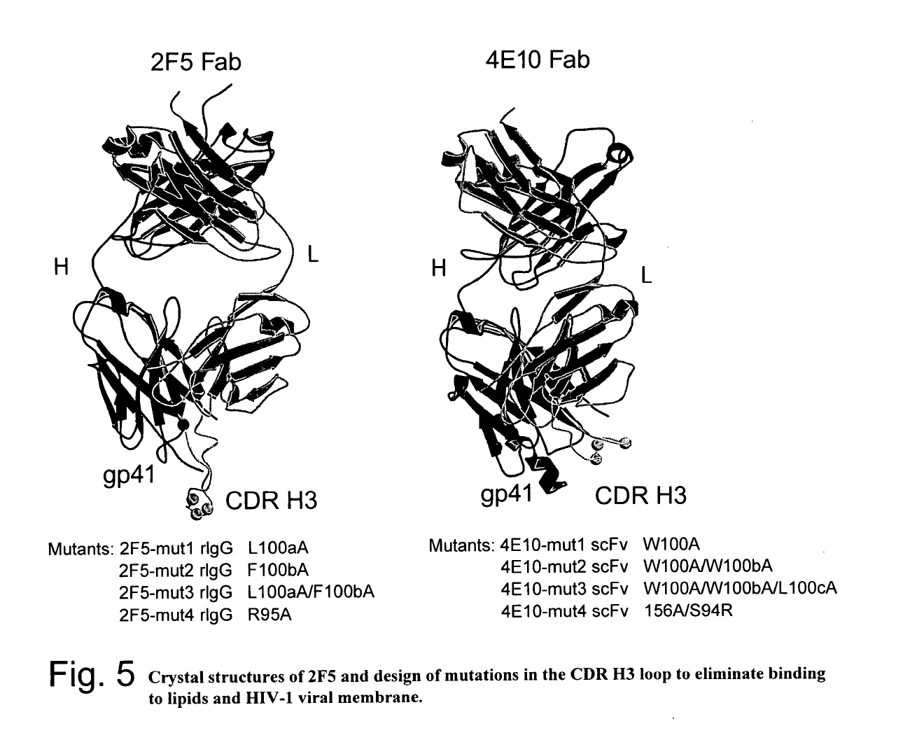

Toll-like receptor ligands, shown in FIG. 2, were formulated in liposomal forms with gp41 MPER peptide immunogens or gp41-inter protein (FIG. 1 and FIG. 3 (Frey et al, Proc. Natl. Acad. Sci. 105:3739-3744 (2008)). The structures in FIG. 2 are examples only and other forms of TLR agonists (Takeda et al, Annu. Rev. Immunol., 21:335-376 (2003)) can be incorporated into similar liposomes as well. A preferred combination of TLR agonists to be used in the present constructs is oCpG (TLR9; Hemni et al., 2004, Nature, 408:740-745) and R848 (TLR9; Hemni et al, Nat. Immunol., 2002).

The construction of Lipid A and R-848 containing MPER peptide liposomes utilized the method of co-solubilization of MPER peptide having a membrane anchoring amino acid, sequence and synthetic lipids 1-Palmitoyl-2-Oleoyl-sn-Glycero-3-Phosphocholine (POPC), 1-Palmitoyl-2-Oleoyl-sn-Glycero-3-Phosphoethanolamine (POPE), 1,2-Dimyristoyl-sn-Glycero-3-Phosphate (DMPA) and Cholesterol at mole fractions 0.216, 45.00, 25.00, 20.00 and 1.33 respectively (Alam et al, J. Immunol. 178:4424-4435 (2007)). Appropriate amount of MPER peptide dissolved in chloroform-methanol mixture (7:3 v/v), Lipid A dissolved in Chloroform or R-848 dissolved in methanol, appropriate amounts of chloroform stocks of phospholipids were dried in a stream of nitrogen followed by over night vacuum drying. Liposomes were made from the dried peptide-lipid film in phosphate buffered saline (pH 7.4) using extrusion technology.

Construction of oligo-CpG complexed MPER peptide liposomes used the cationic lipid 1-Palmitoyl-2-Oleoyl-sn-Glycero-3-ethylphospho choline (POEPC) instead of POPC. Conjugation of oCpG was done by mixing of cationic liposomes containing the peptide immunogen with appropriate amounts of oCpG stock solution (1 mg/ml) for the desired dose.

Surface Plasmon Resonance (SPR) assay for the binding of 2F5 mAb to its epitope in the MPER 2F5 peptide epitope-liposome constructs revealed that incorporation or conjugation of TLR adjuvants does not affect binding of HIV neutralizing antibody 2F5 to gp41 peptide in liposomes. Strong binding of both mAbs 2F5 and 4E10 was observed in the peptide lipsome constructs described in FIG. 4.

### EXAMPLE 2

Autoreactive B cells can be activated by TLR ligands through a mechanism dependent on dual engagement of the BCR and TLR (Leadbetter et al, Nature 416:603 (2002); Marshak-Rothstein et al, Annu. Rev. Immunol. 25:419-41 (2007), Herlands et al, Immunity 29:249-260 (2008), Schlomchik, Immunity 28:18-28 (2008)). In this immunogen design, soluble IFN-α has been encapsulated into liposomes conjugated to either MPER656 or MPER656-L669S peptides. IFN-α has been reported to modulate and relax the selectivity for autoreactive B cells by lowering the BCR activation threshold (Uccellini et al, J. Immunol. 181:5875 (2008)). The design of these immunogens is also based on the observation that lipid reactivity of gp41 MPER antibodies is required for both binding to membrane bound MPER epitopes and in the neutralization of HIV-1.

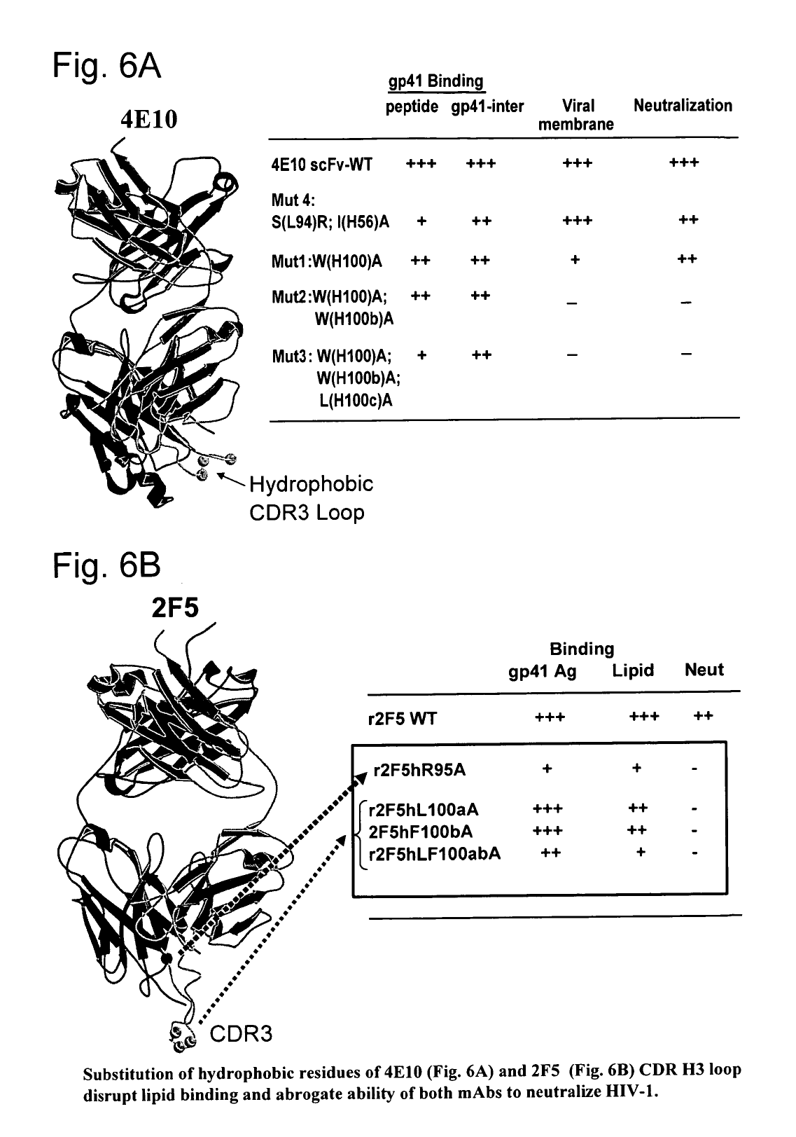

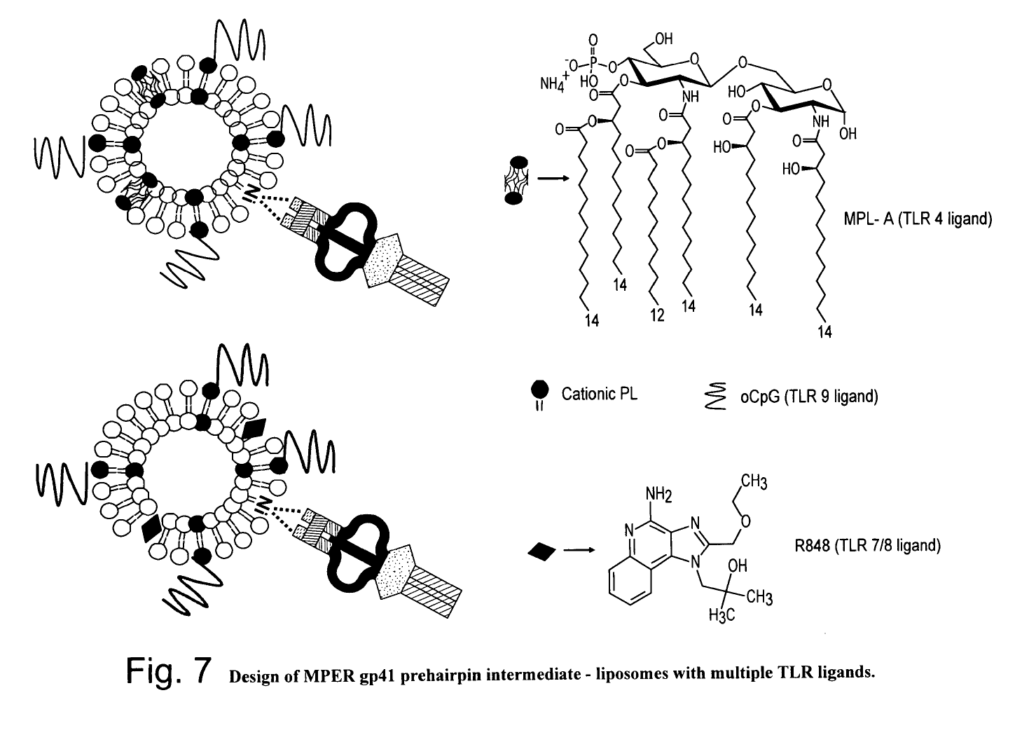

The long CDR H3 loops of MPER neutralizing mAbs 4E10 and 2F5 have a hydrophobic face, postulated to interact with virion membrane lipids (Ofek et al, J. Virol. 78:10724 (2004); Cardoso et al, Immunity 22:163-173 (2005)). CDRH3 mutants of 4E10 (scFv) and 2F5 (IgG) have been constructed (FIG. 5) and it has been found that binding of neutralizing MPER mAbs occur sequentially and is initiated by binding of mAbs to viral membrane lipids prior to binding to prefusion intermediate state of gp41. 4E10 scFv bound strongly to both nominal epitope peptide and a trimeric gp41 fusion intermediate protein, but bound weakly to both HIV-1 and SIV virions and thus indicating that 4E10 bound to viral membrane lipids and not to the prefusion state of gp41. While alanine substitutions at positions on the hydrophobic face of the CDR H3 loops of 4E10 (W100a/W100b/L100cA) showed similar binding to gp41 epitopes, the same substitutions disrupted the ability of 4E10 to bind to HIV-1 viral membrane (FIG. 6). 4E10 CDR H3 mutants that bound to gp4l intermediate protein but did not bind to HIV-1 viral membrane failed to neutralize HIV-1. Similarly, 2F5 CDR H3 mutants with disruptions in binding to HIV-1 virions but not to gp41 epitope peptide, failed to neutralize HIV-1 (FIG. 6). Blocking of HIV-1 neutralization activity of 4E10 by gp41 fusion intermediate protein further suggested that 4E10 did not bind to viral prefusion gp41. These results support the model that binding of neutralizing MPER mAbs occurs sequentially and is initiated by binding of mAbs to viral membrane lipids prior to binding to prefusion intermediate state of gp41. An important implication of this result is that the HIV-1 membrane constitutes an additional structural component for binding and neutralization by 4E10 and 2F5. Thus, a lipid component may be required for an immunogen to induce 4E10 and 2F5- like antibody responses.

Thus, this strategy has the potential to modulate B cell tolerance, target immunogens to responsive B cell subsets, and allow the induction of polyreactive B cells that bind to phospholipids and gp4l MPER epitopes. When used in combination with TLR ligands, the delivery of IFN-α in liposomes has the potential to allow TLR-dependent activation of B cells from the autoreactive pool and with the desired specificity for gp41 MPER epitopes.

### Description of Constructs

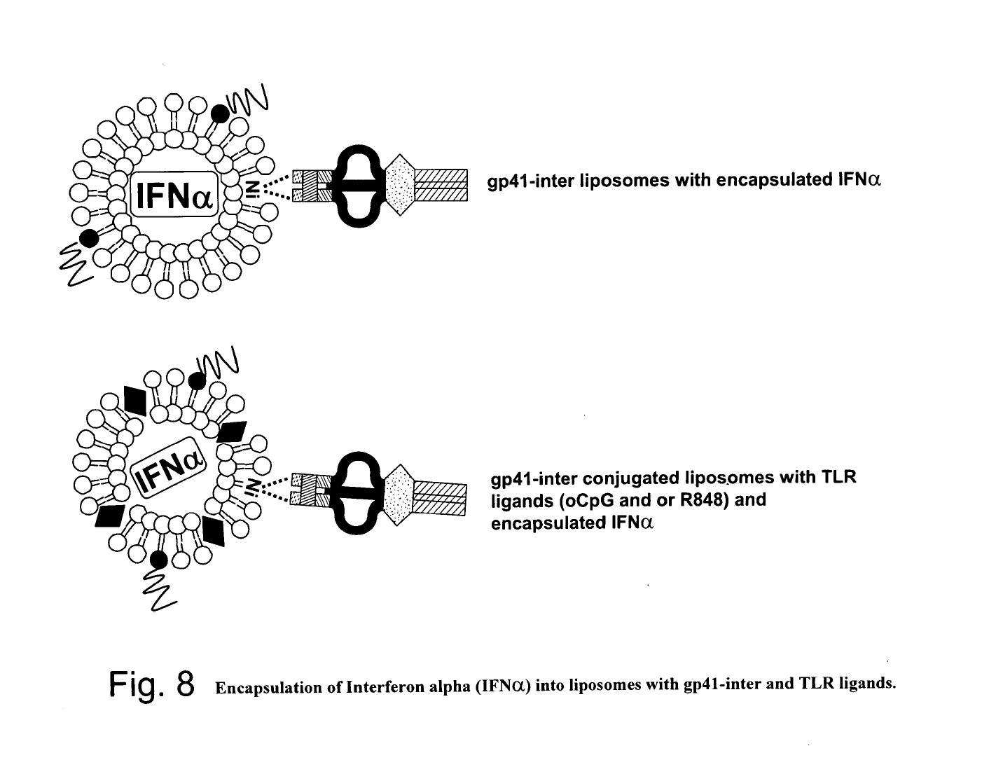

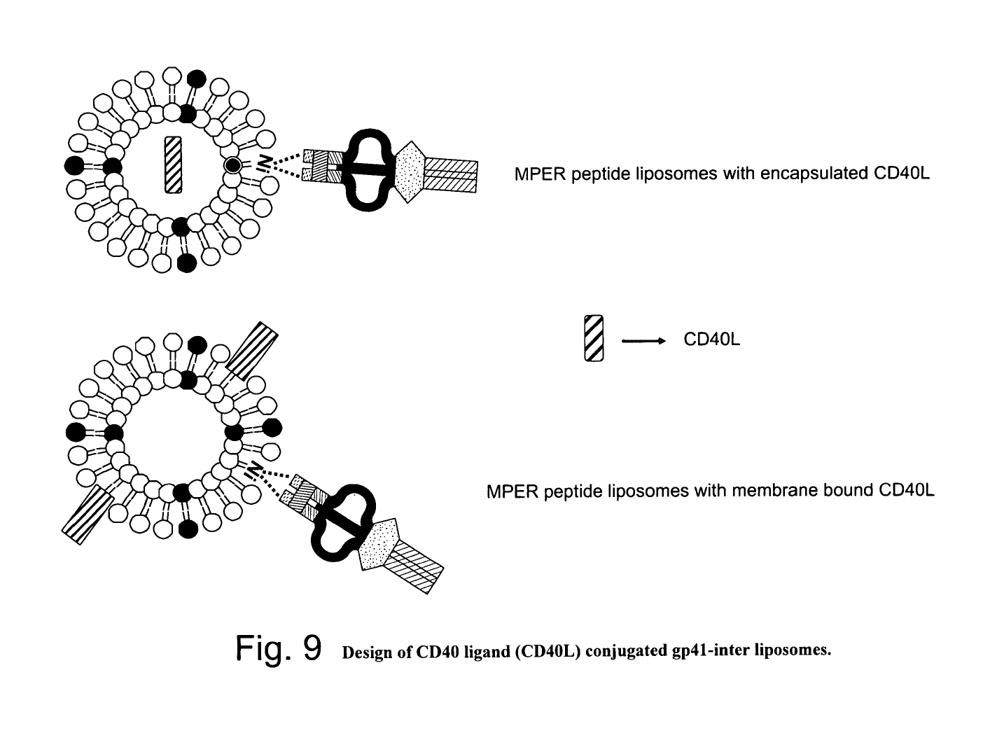

The HIV-1 gp41 MPER gp41 intermediate construct (FIG. 1) can be conjugated to synthetic liposomes as outlined above. Each of the sonicated MPER gp41 intermediate construct-liposomes (FIGS. 7 and 8) can be prepared and then mixed with soluble IFNα protein and then dried and rehydrated to encapsulate the cytokine. After brief vortexing, the rehydrated liposomes with encapsulated IFNα can be collected by ultracentrifugation for 30 min. In the first design, liposome is conjugated to either oCpG (TLR 9), MPL-A (TLR4) or R848 (TLR7/9) (FIGS. 2 and 3). Each of these adjuvanted liposome constructs can be prepared with a form of the gp41 prehairpin intermediate as shown in FIG. 3. A second design is shown in FIGS. 7 and 8 and includes multiple TLR ligands, TLR 9+TLR 4 and TLR9+TLR 7/8 incorporated into the same liposomes. The design of these constructs can provide synergy in TLR triggering and could potentially enhance the potency of the TLR ligands in activating polyreactive B cells. Additionally, designed constructs have been designed with either soluble CD40L or membrane bound CD40L incorporated with gp41-inter liposomes as shown in FIG. 9.

The assessment of the presentation of MPER epitopes on the adjuvanted liposome constructs can be done by SPR analysis of 2F5 and 4E10 mAb binding as described in FIG. 4.

### EXAMPLE 3

### Experimental Details

Ni-NTA (N″,N″-bis[carboxymethyl]-L-lysine; nitriloacetic acid, NTA) liposomes were constructed from synthetic lipids POPC, POPE, DOGS (1,2 dioleoyl-sn- glycerol-3-succinyl-NTA-Ni) and cholesterol at mole fractions 45, 25, 5 and 25 respectively using methods described earlier (Alam et al., J. Immunol. 178:4424-4435 (2007)). Conjugation of His tagged gp41-inter to the Ni-NTA liposomes was verified by surface plasmon resonance experiment. The His tagged gp41-inter when injected over the immobilized liposomal surfaces bound selectively to the Ni-NTA liposomes when compared to the control liposomes that lacked Ni-NTA. The presentation of epitopes of MPER neutralizing antibodies in the liposome conjugated gp41-inter was examined by comparing the binding of 2F5 and 4E12 mAbs to the gp41-inter bearing Ni-NTA liposomes with that of unconjugated Ni-NTA liposomes. Both 2F5 and 4E10 mAbs bound selectively to the gp41-inter bearing Ni-NTA liposomes

### Results

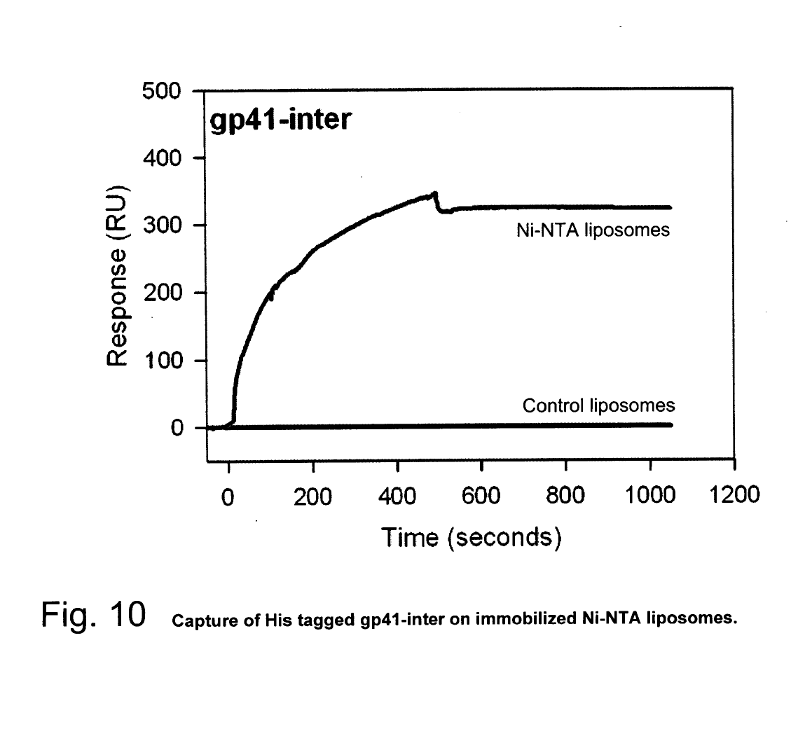

FIG. 10 shows capture of His tagged gp41-inter on immobilized Ni-NTA liposomes. HIV-1 gp41-inter with a short sequence of histidine residues (His6) at the c-terminus end (described in FIG. 1) was immobilized on synthetic liposomes containing a nickel-chelating group (N″,N″-bis[carboxymethyl]-L-lysine; nitriloacetic acid, NTA) covalently attached to the lipid molecules (DOGS, 1,2 dioleoyl-sn-glycerol-3-succinyl-NTA-Ni). SPR binding assay shows shows specific capture of gp41-inter to Ni-NTA liposomes but not to control liposomes lacking Ni-NTA. The slow dissociation of gp41-inter is indicative of stable immobilization of gp41-inter to liposomes.

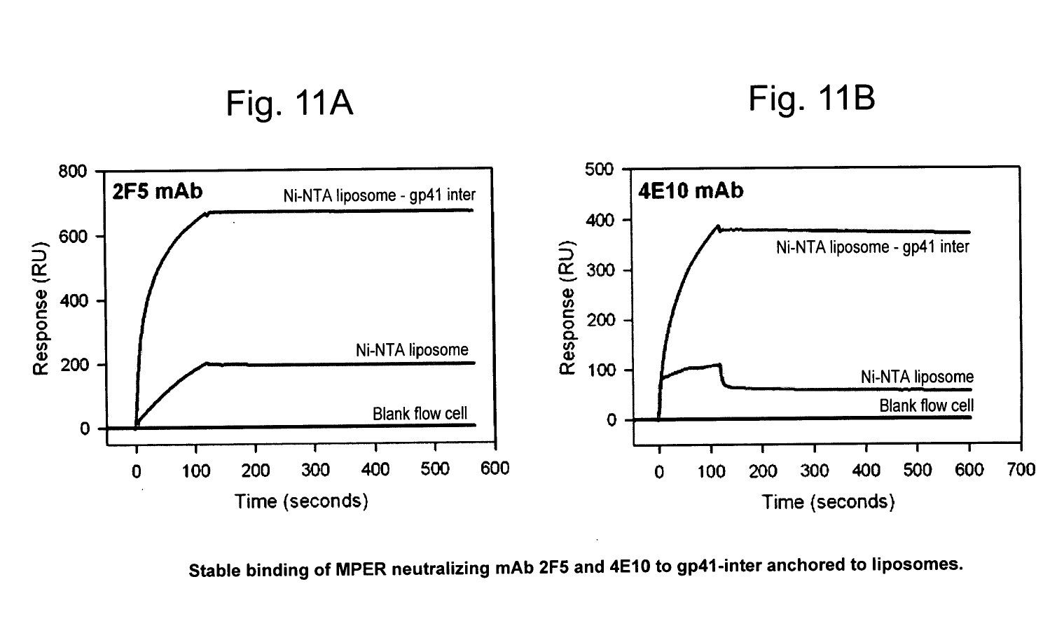

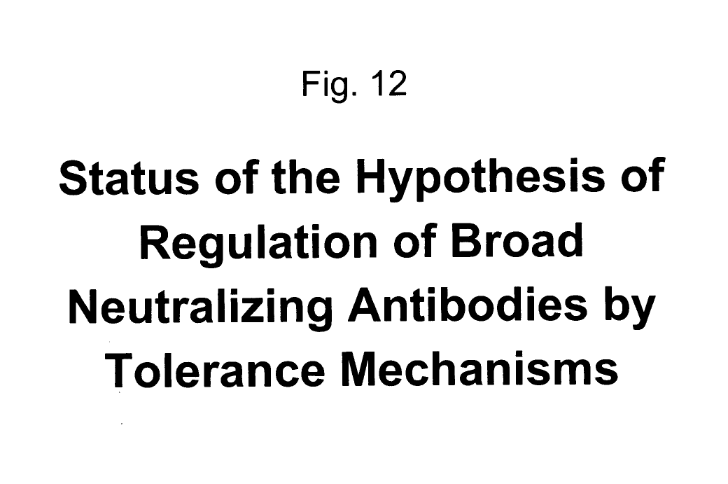

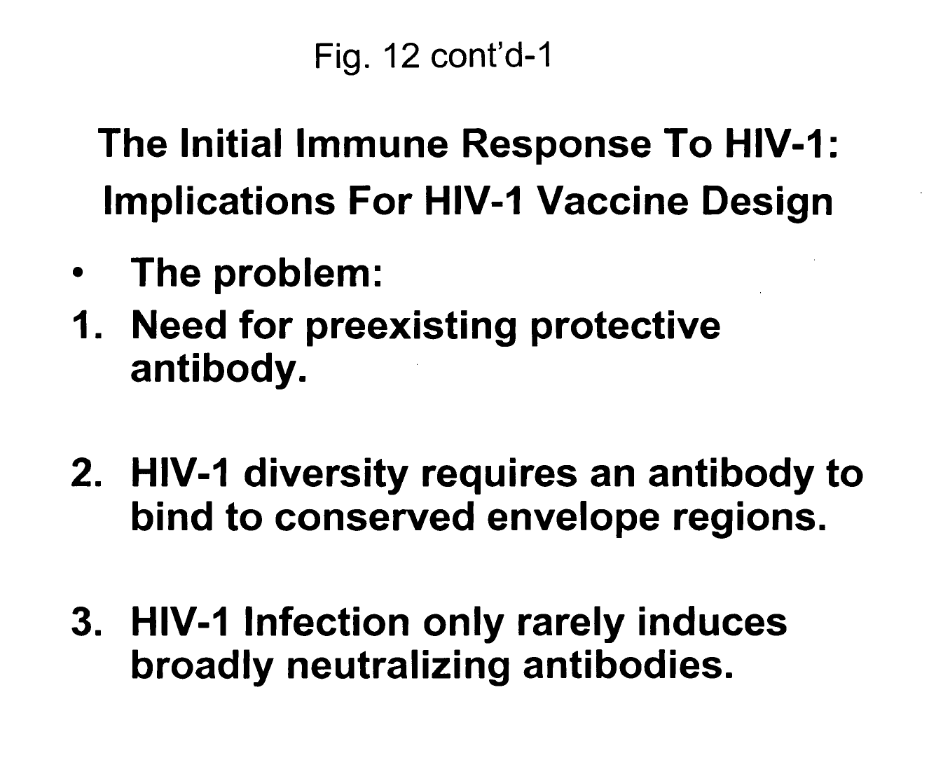

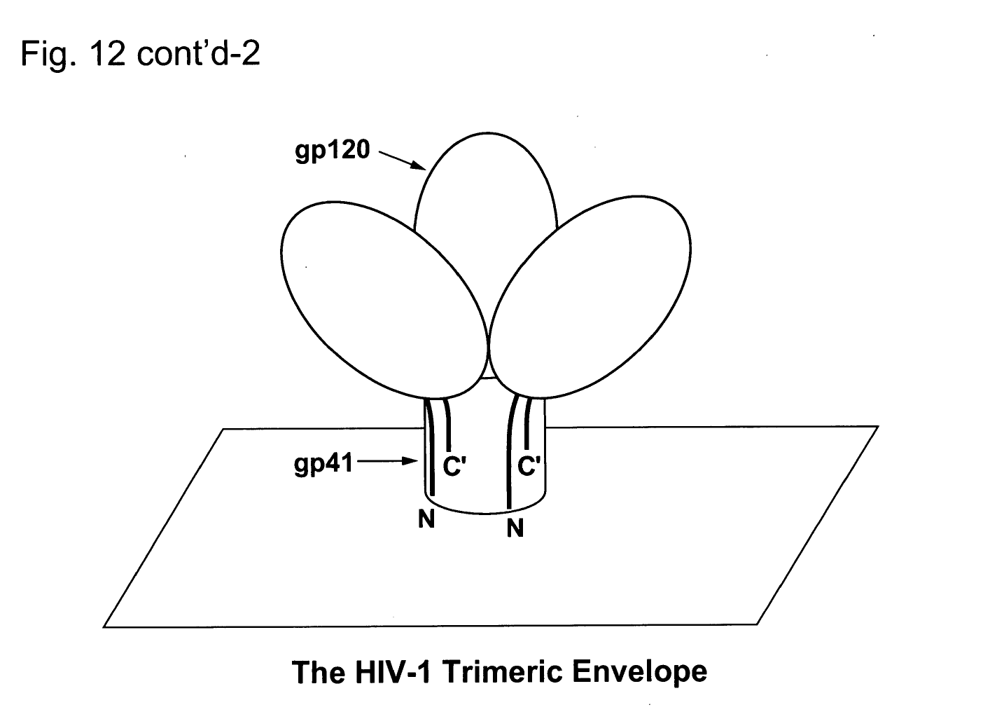

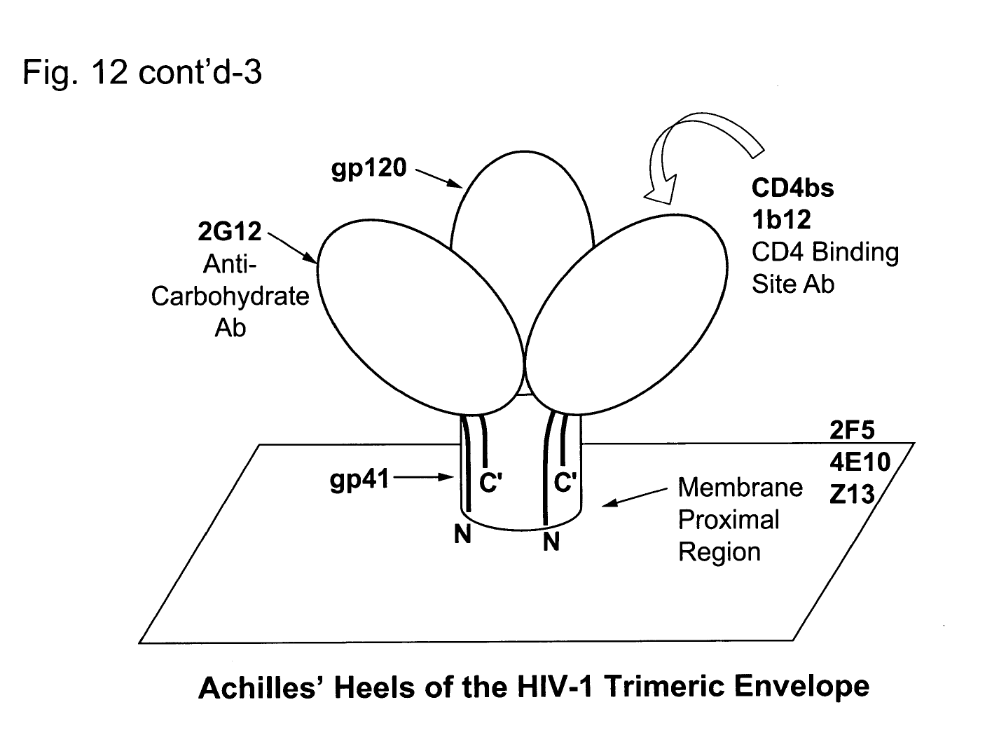

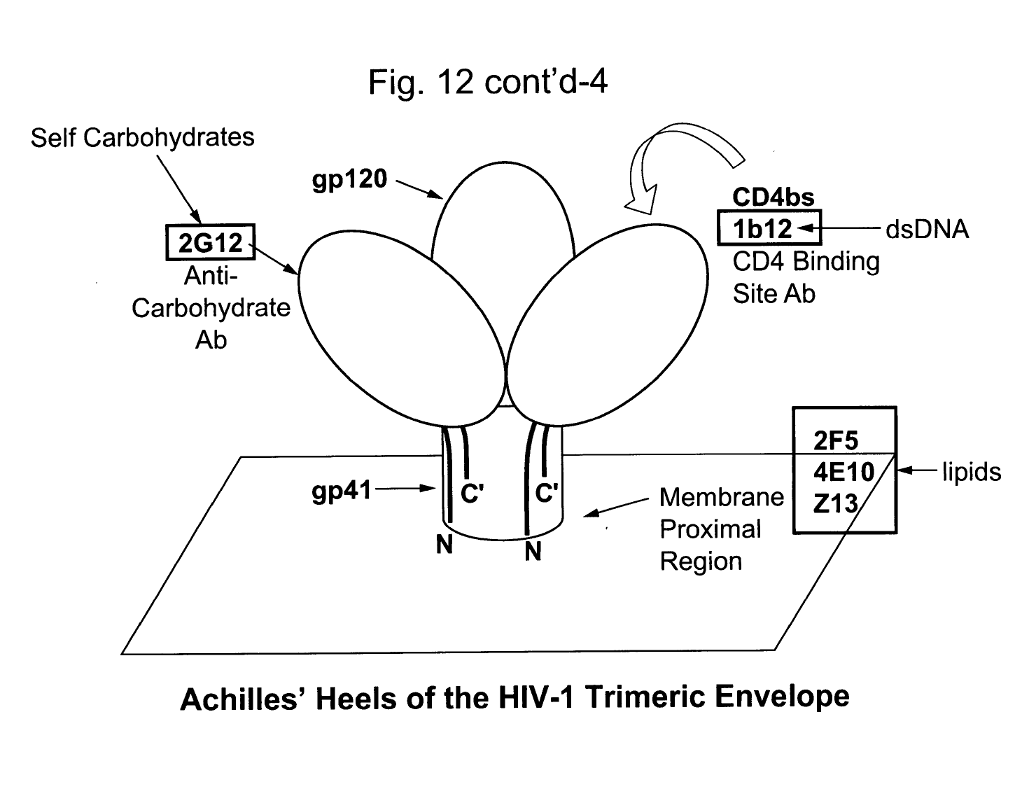

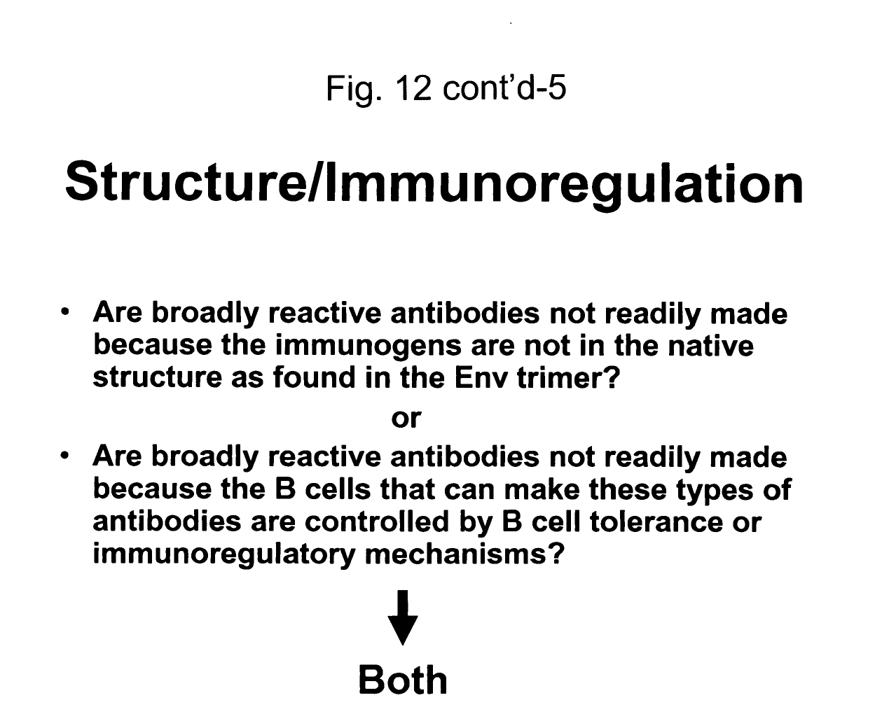

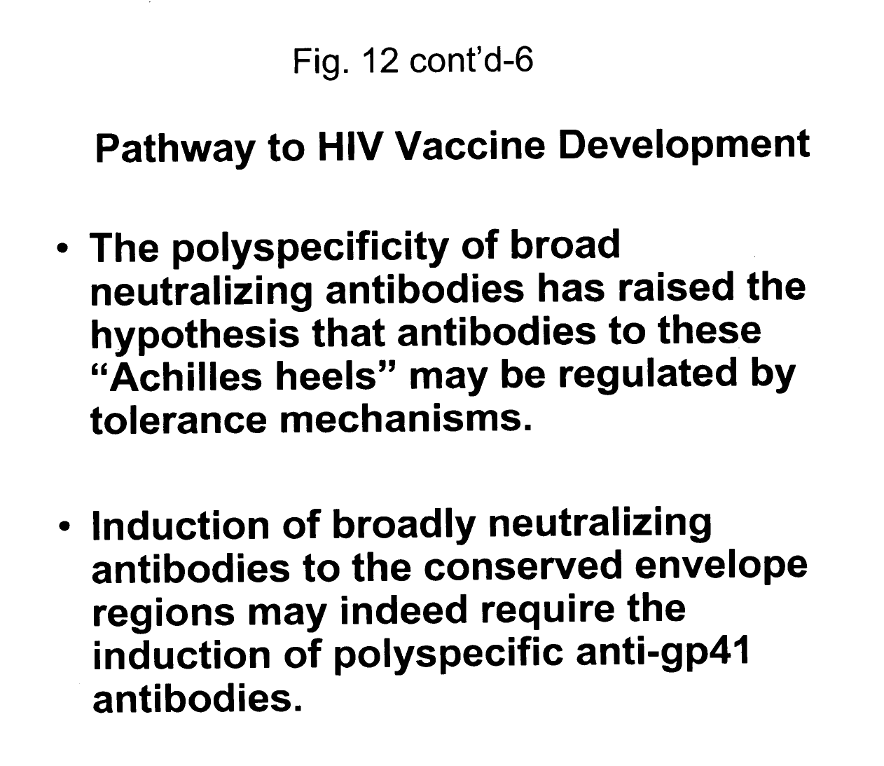

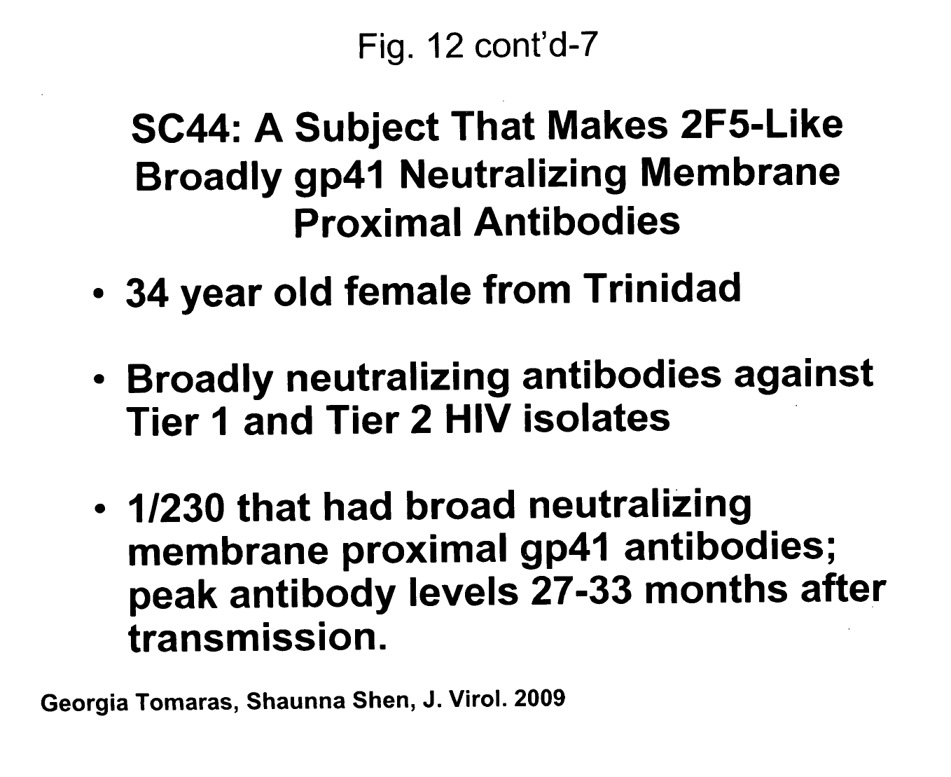

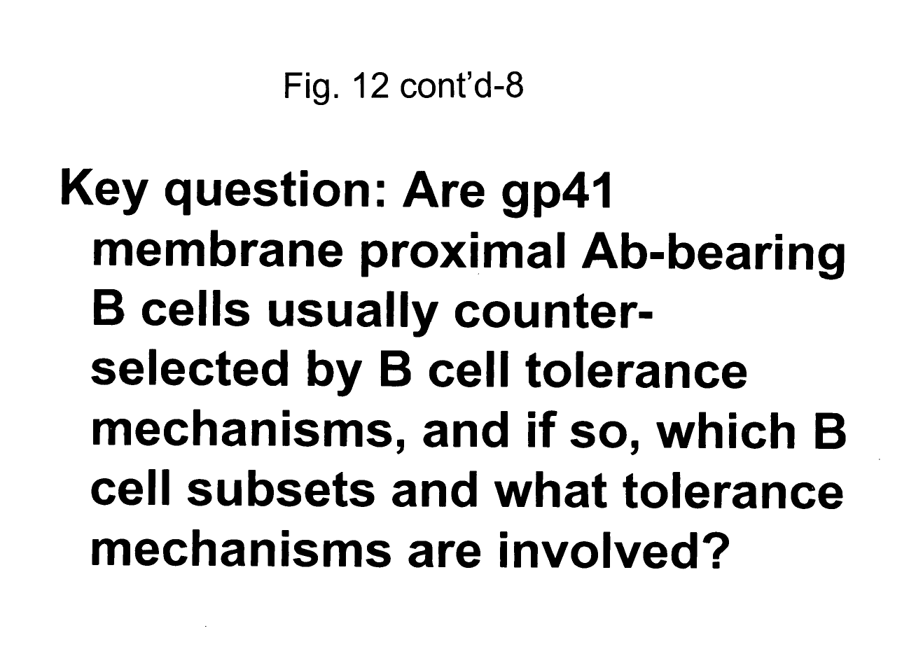

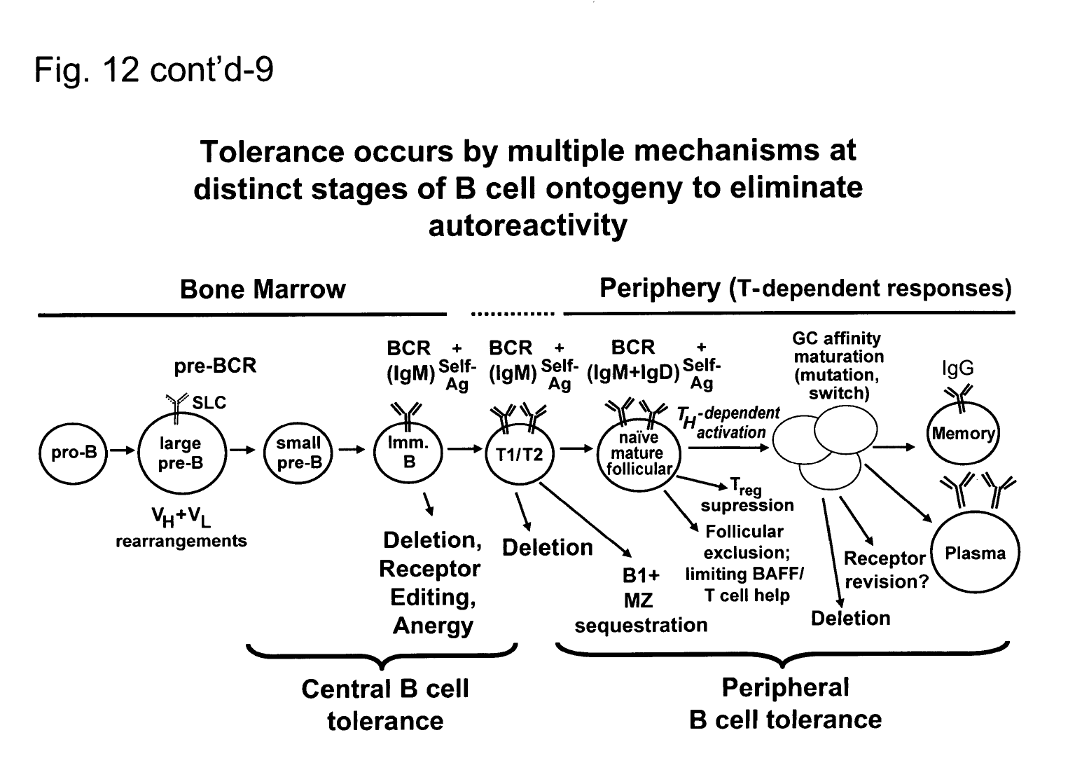

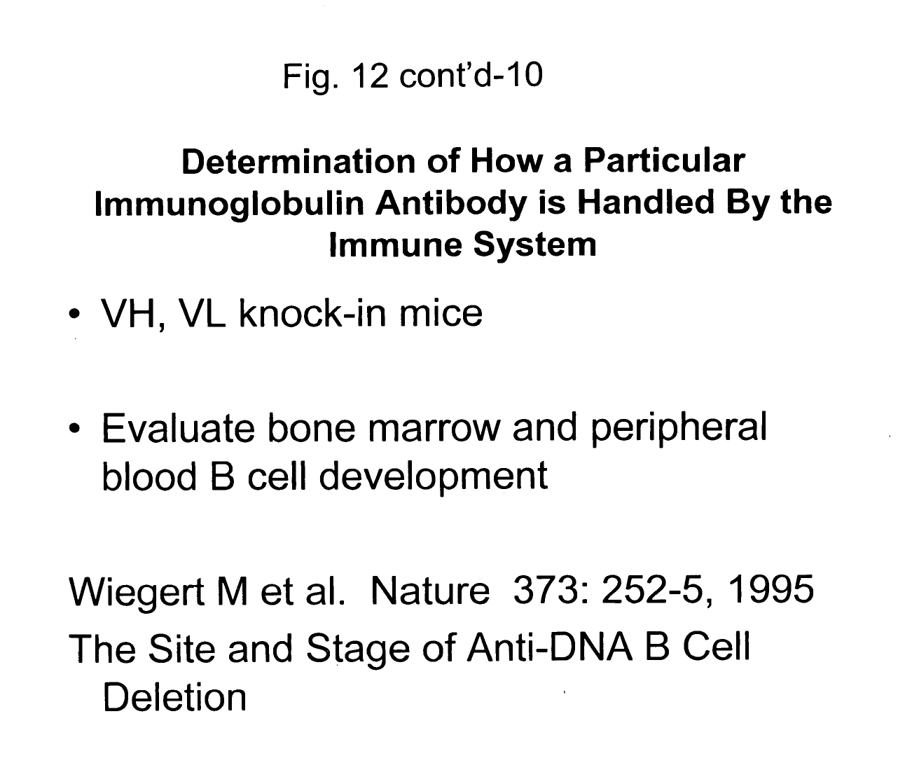

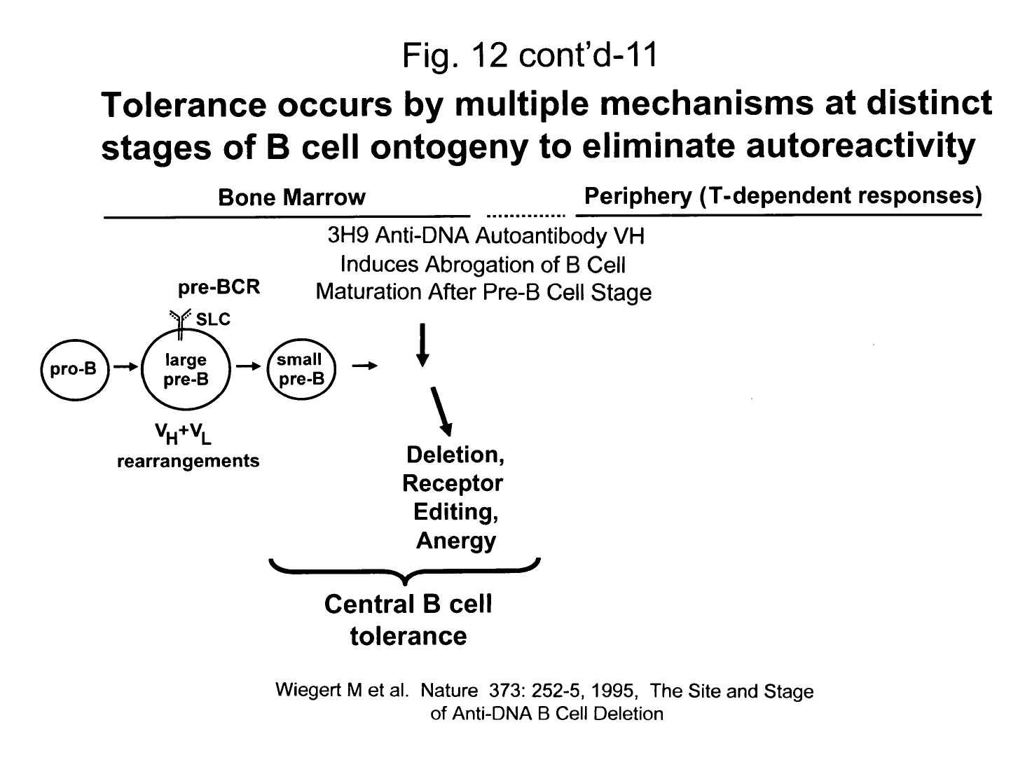

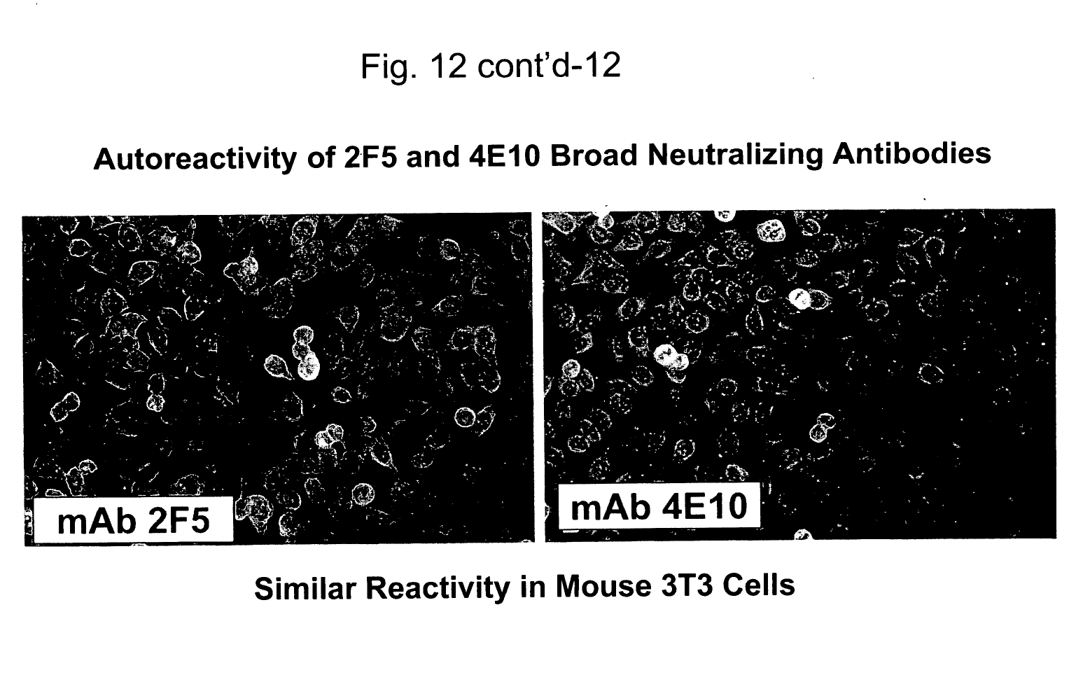

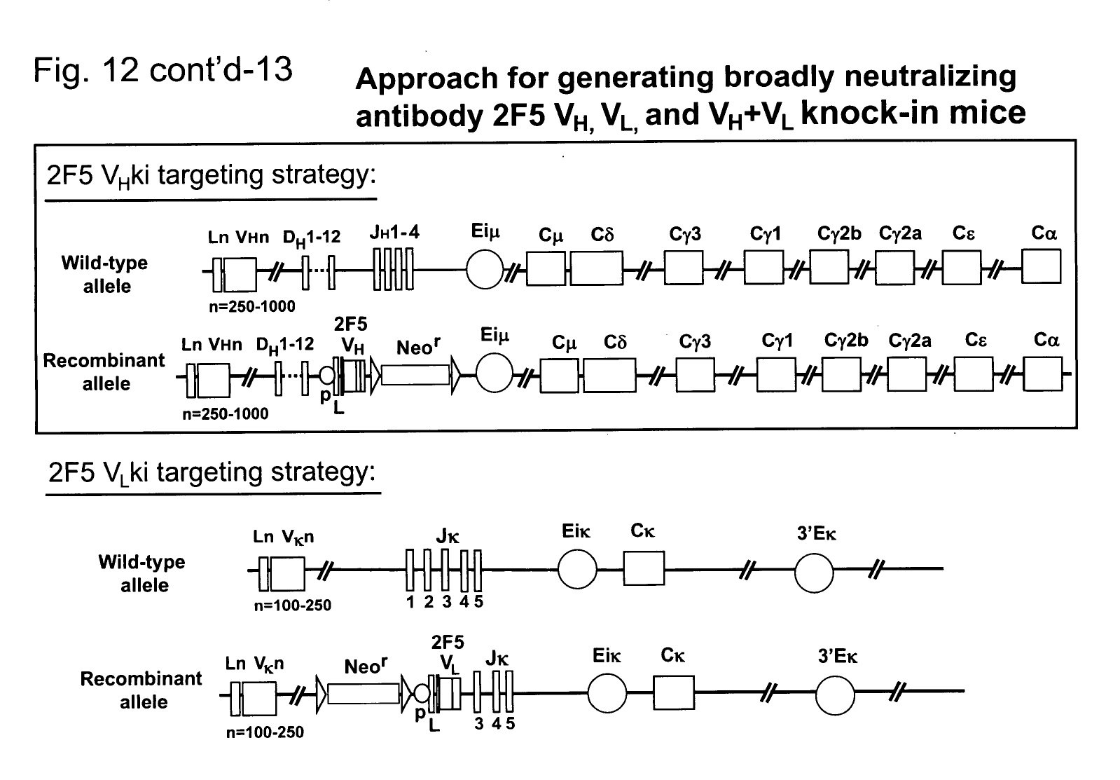

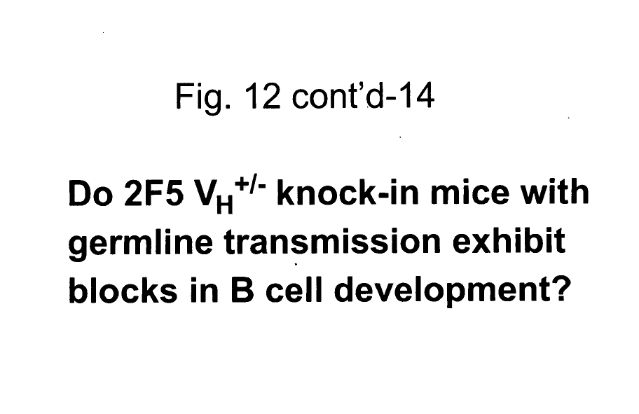

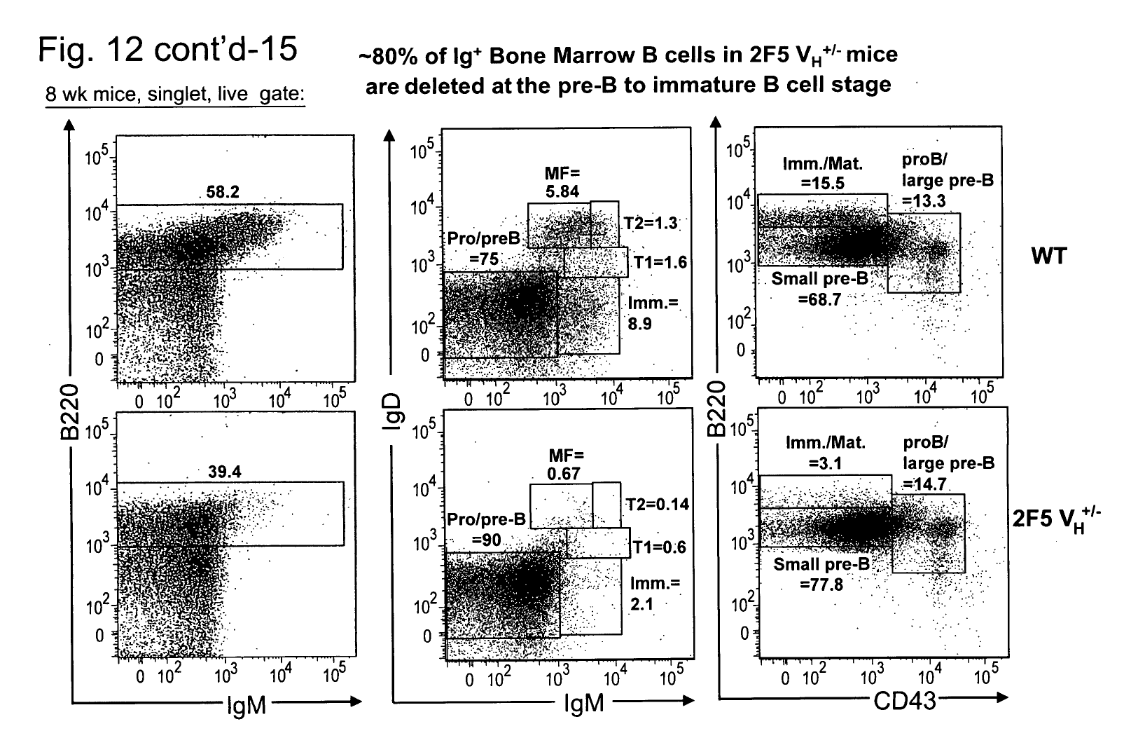

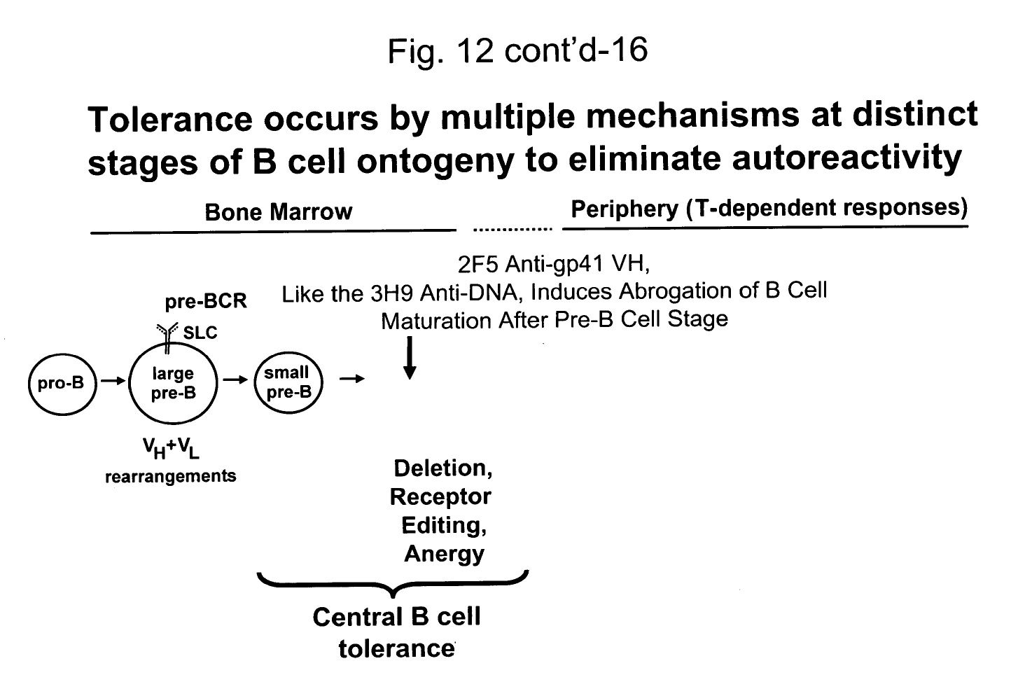

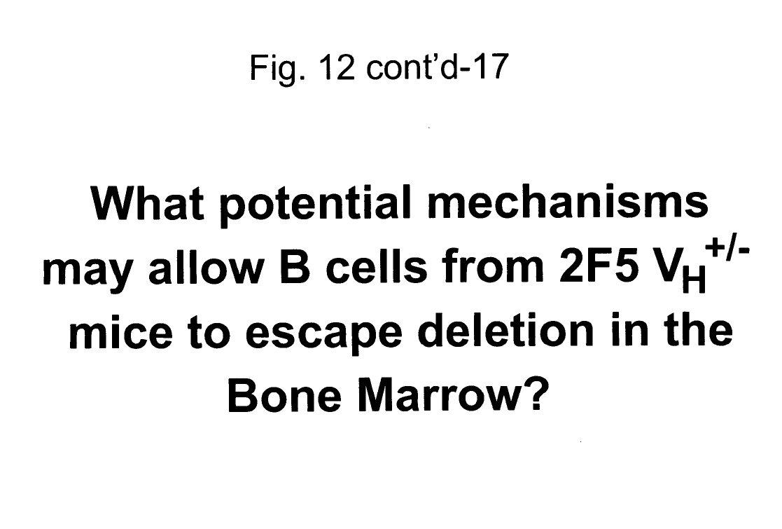

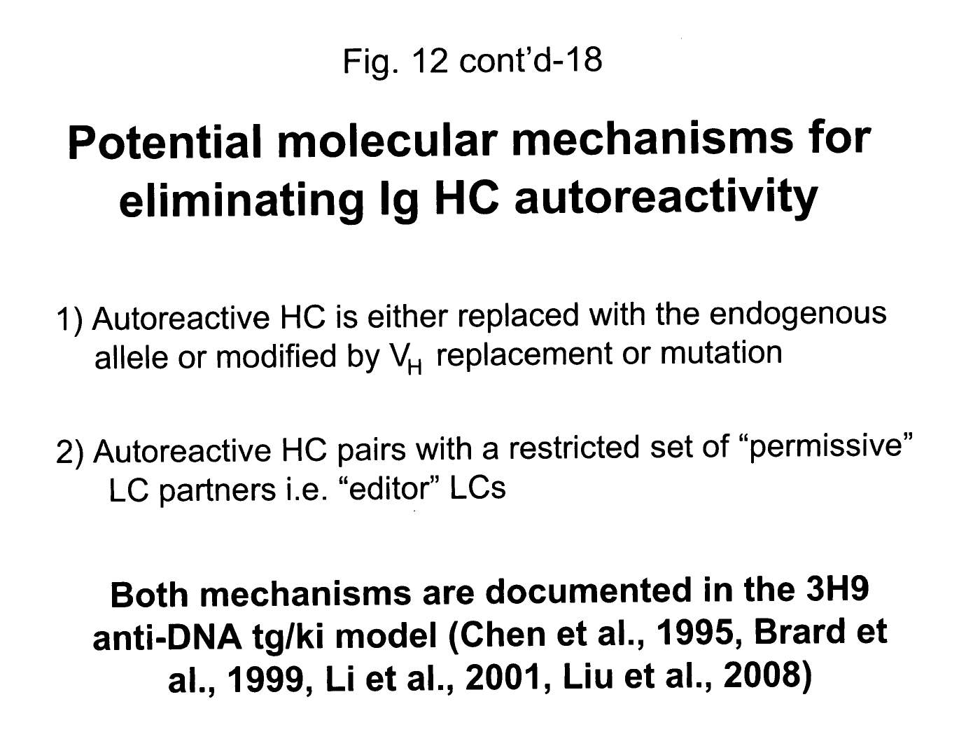

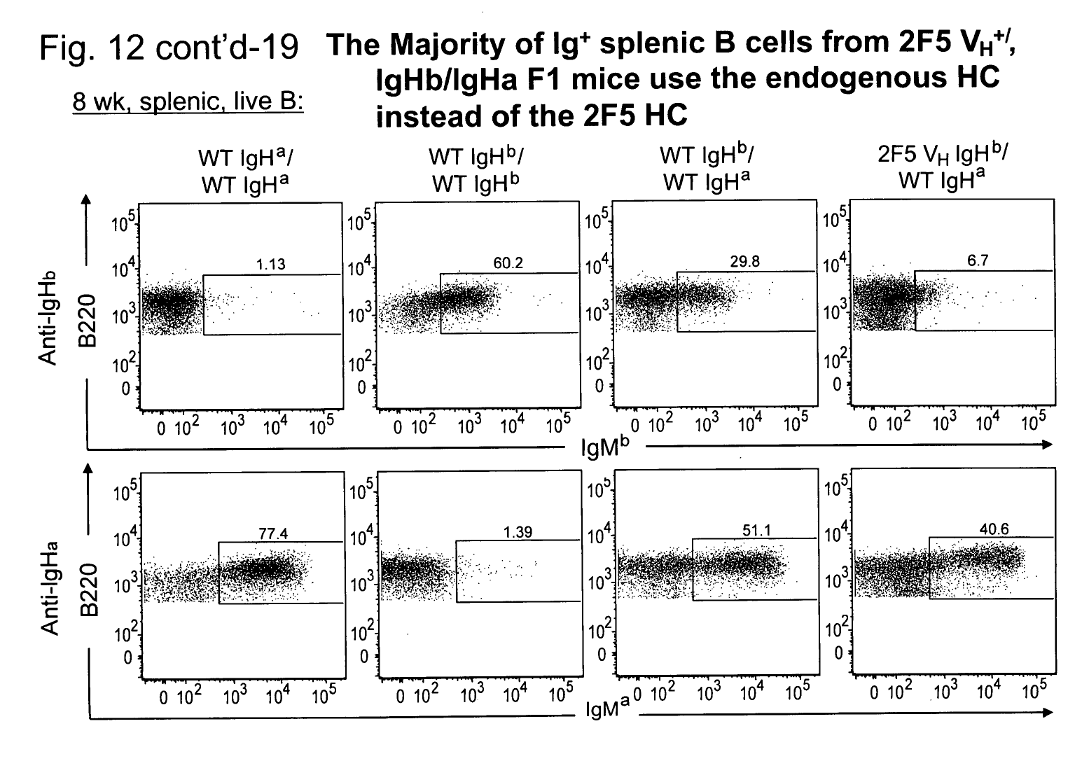

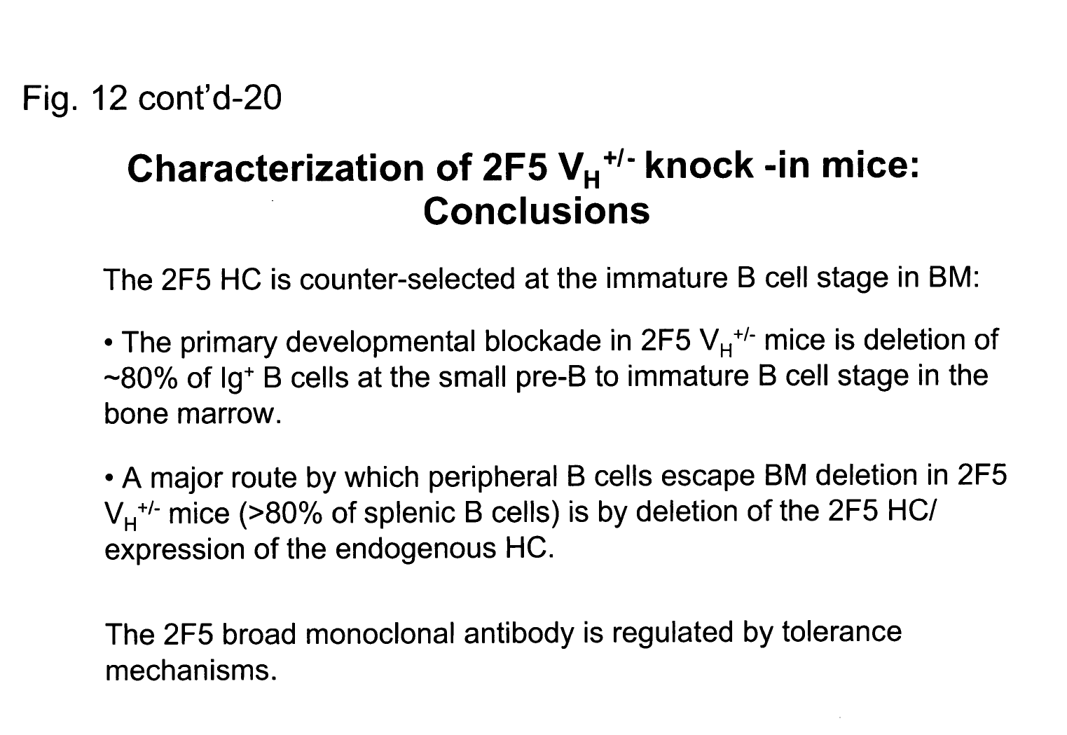

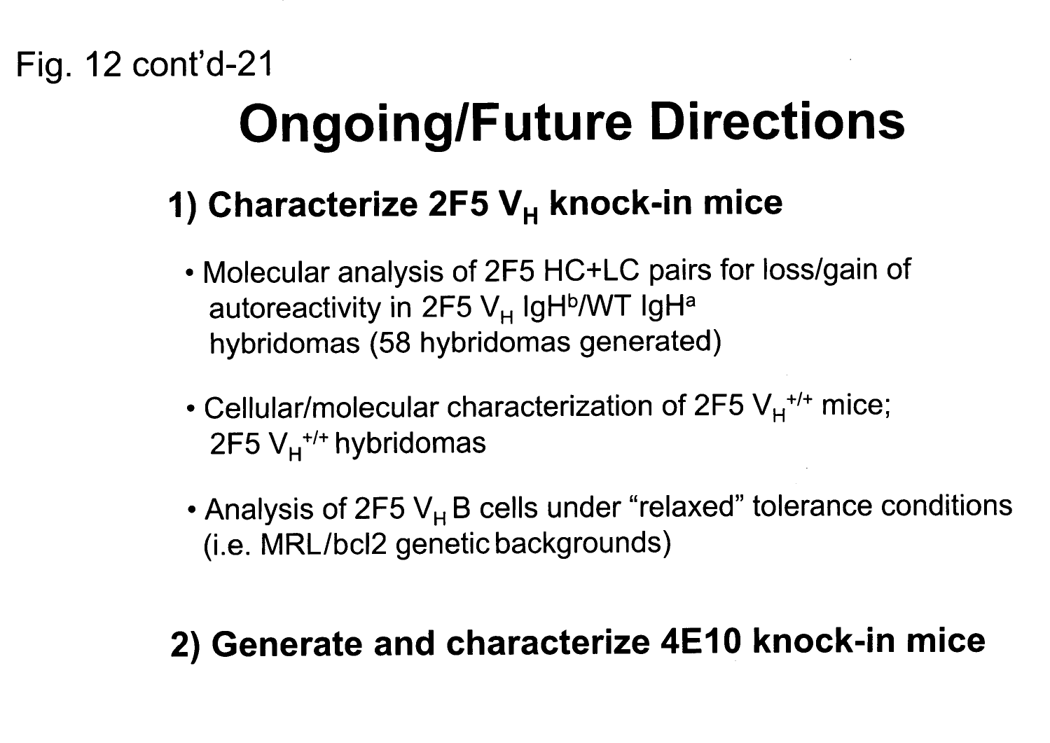

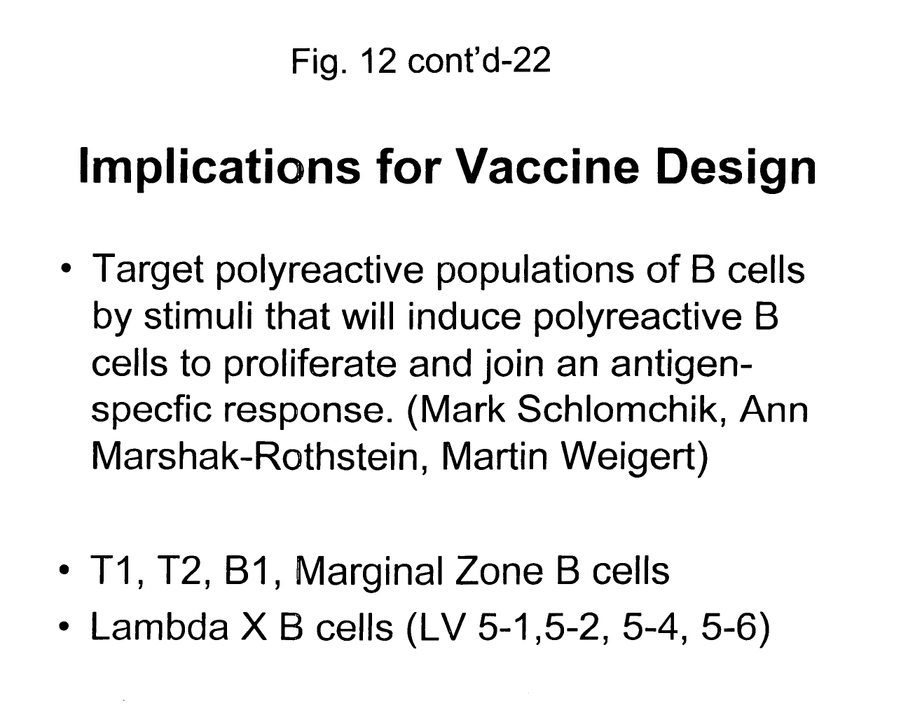

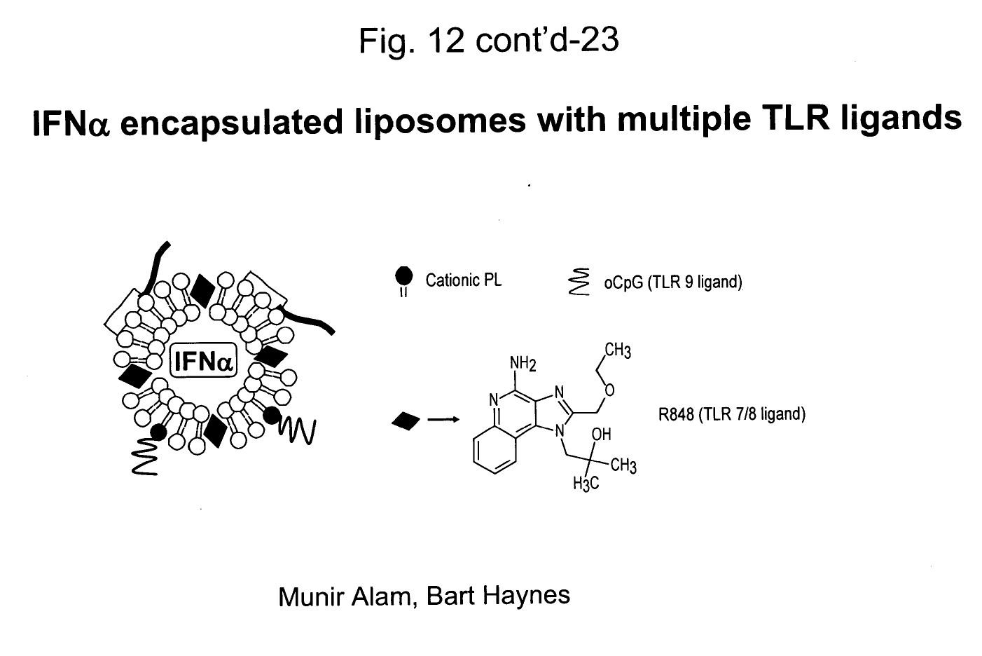

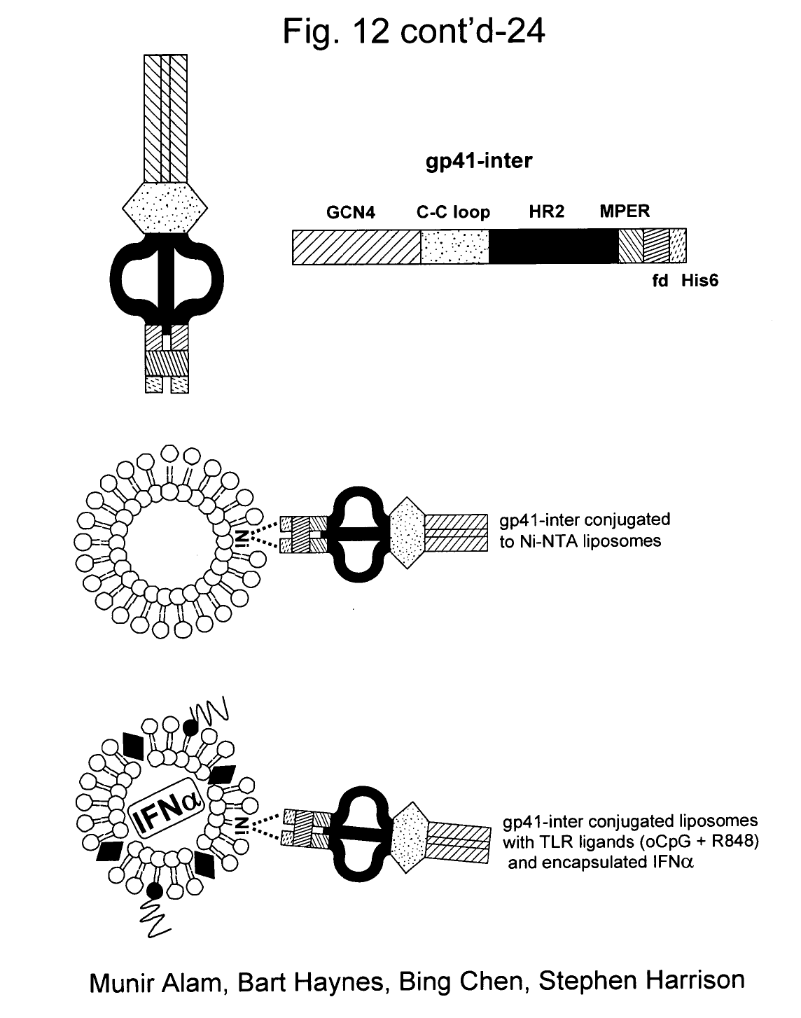

FIG. 11 shows stable binding of MPER neutralizing mAb 2F5 and 4E 10 to gp41-inter anchored to liposomes. gp41-inter protein was anchored to Ni-NTA-liposomes and followed by injection of 2F5 mAb (A, 50 ug/mL) and 4E10 mAb (B, 50 μg/ml). Strong binding of both 2F5 and 4E10 mAbs to gp41-inter-liposomes was observed. Background binding to the controls, Ni-NTA liposomes without gp41 protein and sensor surface (blank flow cell) are also shown. Binding of both 2F5 and 4E10 mAbs show much slower dissociation rates when compared to those of MPER peptide-lipid conjugates. These data show that gp41-inter can form stable complexes with Ni-NTA liposomes and the MPER epitopes on the trimeric gp41-inter are optimally presented for high affinity binding to 2F5 and 4E 10 mAbs. This lays the foundation for anchoring gp41-inter protein to TLR adjuvants and cytokine (TNF-a) conjugated liposomes and to be used as immunogens for the induction of polyreactive and broadly neutralizing MPER mAbs

All documents and other information sources cited above are hereby incorporated in their entirety by reference.

What is claimed is:

1. A method of inducing in mammals of broadly neutralizing anti-HIV-1 antibodies with gp41-lipid constructs comprising a prehairpin intermediate form of HIV-1 envelope gp41 linked to a synthetic liposomes.

2. The method of claim 1 wherein said liposome comprises a TLR agonist.

3. The method of claim 2 wherein said TLR agonist is specific for TLR 7/8 or TLR 9.

4. The method of claim 2 wherein said TLR agonist is specific for TLR 4 or TLR5.

5. The method of claim 1 wherein IFNa is incorporated into said liposome.
